(19) United States
(12) Patent Application Publication
(10) Pub. No.: US 2013/0045895 A1
(43) Pub. Date: Feb. 21, 2013

(54) BINDING DOMAINS

(71) Applicants: Rudolf Maria De Wildt, Stevenage (GB); Mark Liddament, Cambridge (GB); Nicola Ramsay, Cambridge (GB); Oliver Schon, Cambridge (GB); Adriaan Allart Stoop, Cambridge (GB)

(72) Inventors: Rudolf Maria De Wildt, Stevenage (GB); Mark Liddament, Cambridge (GB); Nicola Ramsay, Cambridge (GB); Oliver Schon, Cambridge (GB); Adriaan Allart Stoop, Cambridge (GB)

(21) Appl. No.: 13/642,200

(22) PCT Filed: Apr. 19, 2011
(86) PCT No.: PCT/EP2011/056220

§ 371 Date: Oct. 19, 2012

Related U.S. Application Data
(60) Provisional application No. 61/326,258, filed on Apr. 21, 2010.

(51) Int. Cl. C07K 16/00; C40B 40/08; C07H 21/04; C40B 40/10

(52) U.S. Cl. 506/17; 530/387.3; 506/18; 536/23.53

(57) ABSTRACT

The invention relates to amino acid residues within an immunoglobulin light chain amino acid sequence (V_{L}) which stabilize the monomeric state of the immunoglobulin single variable domain. In particular, but not exclusively, the invention describes a number of mutations that stabilize the monomeric state of DP_{K}9 framework V_{κ} domain antibodies.

### FIELD OF THE INVENTION

The invention relates to amino acid residues within an immunoglobulin light chain amino acid sequence (V_{L}) which stabilize the monomeric state of the immunoglobulin single variable domain. In particular, but not exclusively, the invention describes a number of mutations that stabilize the monomeric state of DP_{K}9 framework V_{κ} domain antibodies.

### BACKGROUND OF THE INVENTION

Domain antibodies are the smallest known antigen-binding fragments of antibodies comprising the robust variable regions of the heavy or light chains of immunoglobulins (V_{H} and V_{L}, respectively) (reviewed, for example, in Holt et al. (2003) Trends in Biotechnology Vol. 21, No. 11 p. 484-490).

A number of domain antibodies, including human antibody light and heavy chain variable domain antibodies (Vκ and V_{H} dAbs), camelid V_{H}H domains (nanobodies) and shark new antigen receptors, that bind to specific target molecules/antigens are being developed as immunotherapeutics (see, for example, Enever et al. Current Opinion in Biotechnology (2009); 20: 1-7).

Development of a domain antibody as an immunotherapeutic follows the same approach that has been established in the case of single chain Fvs and involves screening a dAb phage display library to select for target binding polypeptides, followed by affinity maturation to improve antibody affinity (K_{D}). Suitable methods are described, for example in WO 2005/118642.

One of the properties of domain antibodies is that they can exist and bind to target in monomeric or multimeric (especially dimeric) forms. A monomer dAb may be preferred for certain targets or indications where it is advantageous to prevent target cross-linking (for example, where the target is a cell surface receptor such as a receptor tyrosine kinase e.g. TNFR1). In some instances, binding as a dimer or multimer could cause receptor cross-linking of receptors on the cell surface, thus increasing the likelihood of receptor agonism and detrimental receptor signaling. Alternatively, a dAb which forms a dimer may be preferred to ensure target cross-linking or for improved binding through avidity effect, improved stability or solubility, for example.

One of the advantages of small fragments such as domain antibodies is that they can be used in combination with other molecules for formatting and targeting approaches. Such targeting approaches include building multidomain constructs for engaging several targets at the same time. For example, a multidomain construct can be made in which one of the domains binds to serum proteins such as albumin. Domain antibodies that bind serum albumin (AlbudAbs™) are described, for example, in WO05/118642 and can provide the domain fusion partner an extended serum half-life in its own right.

For certain targeting approaches involving a multidomain construct, it may be preferable to use a monomer dAb e.g. when a dual targeting molecule is to be generated, such as a dAb-AlbudAb™ where the AlbudAb binds serum albumin, as described above, since dimerizing dAbs may lead to the formation of high molecular weight protein aggregates, for example.

Accordingly, there is a need to be able to tailor populations of immunoglobulins according to need, such that they comprise an increased proportion of monomers or dimers, depending on the application. In this way, libraries which have a higher proportion of monomers or dimers can be chosen from the outset to develop a monomer or dimer dAb for a particular use. This would enable a drug to be tailored for treating a disease more efficaciously. Alternatively, it may also be desirable to change the dimerization state of an existing dAb or “parental” dAb to tailor according to the need.

An ability preferentially to choose to generate a monomer or dimer dAb gives more flexibility when using these dAbs in formatting and, for example, in dual targeting molecules.

### SUMMARY OF THE INVENTION

The present invention describes amino acid residues within an immunoglobulin light chain amino acid sequence (V_{L}) which stabilize the monomeric state of the immunoglobulin single variable domain. In particular, the present invention describes a number of mutations that stabilize the monomeric state of DP_{K}9 framework V_{κ} domain antibodies. Accordingly, the present invention has application in the design of libraries of V_{L} domain antibodies with a high or low proportion of monomers or dimers depending on the desired properties of the required single variable domain immunoglobulin i.e. the mutations can be varied according to whether the monomeric or dimeric state is preferred. Accordingly, the present invention provides a way to isolate an increased number of candidate dAbs with desirable properties.

Accordingly, in a first aspect, the invention provides an isolated polypeptide comprising a variant immunoglobulin light chain single variable domain wherein said variant comprises the amino acid sequence of a framework region encoded by a human germline antibody gene segment and wherein at least one of the amino acids at positions 36, 38, 43, 44, 46 and 87 has been replaced, said positions assigned in accordance with the Kabat amino acid numbering system. The locations of CDRs and frame work (FR) regions within immunoglobulin molecules and a numbering system have been defined by Kabat et al. (Kabat, E. A. et al., Sequences of Proteins of Immunological Interest, Fifth Edition, U.S. Department of Health and Human Services, U.S. Government Printing Office (1991)). In all aspects or embodiments of the invention where amino acid numbering is indicated, positions are assigned in accordance with Kabat.

According to one further aspect of the invention which may be mentioned, there is provided an isolated polypeptide comprising a variant immunoglobulin light chain single variable domain wherein said variant comprises the amino acid sequence of a framework region encoded by a human germline antibody gene segment and wherein at least one of the amino acids at positions 38, 43 and 44 has been replaced, said positions assigned in accordance with the Kabat amino acid numbering system.

In one embodiment, said variant immunoglobulin light chain single variable domain is a V_{L} immunoglobulin light chain single variable domain. In a further embodiment, said variant immunoglobulin light chain single variable domain is a human V_{L} immunoglobulin light chain single variable domain. Suitably, the immunoglobulin light chain single variable domain is a parental V_{L} amino acid sequence which has a framework region encoded by a human germline antibody gene segment and the variant comprises a mutation in at least one of the former interface V_{H} positions 38, 43 or 44. Also suitably, the immunoglobulin light chain single variable domain is a parental V_{L} amino acid sequence which has a framework region encoded by a human germline antibody gene segment and the variant comprises a mutation in at least one of the former interface V_{H} positions 36, 46 or 87.

In one embodiment, the isolated polypeptide or variant is substantially dimeric in solution. It will be appreciated that the term “substantially” used herein means a proportion of the protein showing a mean molar mass as determined by MALLS under standard conditions (see MALLS/Experimental section; PBS buffer, 1 mg/ml protein concentration) at least 10% higher than the theoretical mass up to the molar mass of the dimeric molecule. The varying degree of determined molar mass already indicated the degree and propensity of the dAb protein to dimerise under these conditions. In this embodiment, the variant has at least one of the following amino acids, Q38, A43 or P44. Suitably, the variant immunoglobulin light chain variable domain is substantially dimeric as determined by SEC MALLS. Suitably, the variant which is substantially dimeric in solution having at least one of Q38, A43 or P44 has an immunoglobulin framework region encoded by a human germline antibody gene sequence that is not derived from the human germline sequence DPK9. In one embodiment, the immunoglobulin light chain parental V_{L} sequence is not DOM7h-8 as defined herein.

In another embodiment, the isolated polypeptide or variant is substantially monomeric in solution. In this embodiment, suitably the variant comprises an amino acid sequence in which the amino acid Q38 has been replaced by any of the amino acids R, N, D, E, or G. Suitably, the variant comprises an amino acid sequence in which the amino acid A43 has been replaced by D, I, L, F, T, or W. Suitably, in an embodiment where A43 has been replaced, it is replaced by D. In another embodiment, the variant comprises an amino acid sequence in which the amino acid A43 has been replaced with K, Y or E. Suitably, the variant comprises an amino acid sequence in which the amino acid P44 has been replaced by R, N, D, C, Q, E, H, I, L, K, M, F, T, Y or V. In another embodiment, the variant comprises an amino acid sequence in which the amino acid P44 has been replaced by A. In another embodiment, the variant comprises an amino acid sequence in which the amino acid Y36 has been replaced with A, Q, G, S, T or V. In another embodiment, the variant comprises an amino acid sequence in which the amino acid Y46 has been replaced with R, D, Q, E or F. Suitably, in an embodiment where Y46 has been replaced, it is replaced by D. In another embodiment, the variant comprises an amino acid sequence in which the amino acid Y87 has been replaced with D, C, L or F. Suitably, in an embodiment where Y87 has been replaced, it is replaced by L. In one embodiment, the variant comprises any combination of any of the amino acid replacements in accordance with any of these embodiments, at any two of the six residues, or at three or more residues, such as four, five or six.

In one embodiment of any aspect or embodiment of the invention, the variant immunoglobulin single variable domain is, or is derived from, a V_{L} domain and, suitably, a Kappa lineage V_{L} (Vκ). A number of human Vκ lineages are known. In one embodiment, the V_{L} is a Kappa I lineage V_{L}, suitably the Kappa I lineage, DPK9 as defined herein.

In another embodiment, the isolated polypeptide is an immunoglobulin single variable domain.

In another aspect of the invention there is provided a V_{K} DPK9 immunoglobulin domain characterized in that at least one of positions 36, 38, 43, 44, 46 or 87 has been mutated, said position determined according to Kabat numbering. In another aspect of the invention which may be mentioned there is provided a V_{K} DPK9 immunoglobulin domain characterized in that at least one of positions 38, 43 or 44 has been mutated, said position determined according to Kabat numbering. It will be appreciated that the term “replaced” as used herein refers to an amino acid substitution wherein the particular amino acid of the native V_{K} DPK9 immunoglobulin domain is mutated or substituted to an alternative amino acid. Suitably, position 36 is mutated to an amino acid selected from A, Q, G, S, T or V, said position determined according to Kabat numbering. Suitably, position 38 is mutated to an amino acid selected from R, N, D, E and G said position determined according to Kabat numbering. Suitably, position 43 is mutated to an amino acid selected from D, I, L, F, K, E, T and W said position determined according to Kabat numbering. Suitably, position 44 is mutated to an amino acid selected from R, N, D, C, Q, E, H, I, L, K, M, F, T, Y and V, said position determined according to Kabat numbering. Suitably, position 46 is mutated to an amino acid selected from R, D, Q, E or F, such as D, said position determined according to Kabat numbering. Suitably, position 87 is mutated to an amino acid selected from D, C, L or F, such as L, said position determined according to Kabat numbering. In one embodiment, the V_{K} DPK9 immunoglobulin domain comprises a combination of any two of the amino acid mutations in accordance with any embodiment of the invention. Suitably, a V_{K} DPK9 immunoglobulin domain in accordance with the invention is substantially monomeric in solution. Biophysical properties of a polypeptide or immunoglobulin in accordance with the invention can be measured in accordance with any suitable methods. A number of suitable methods are described herein in the Examples section. In one embodiment, a V_{K} DPK9 immunoglobulin domain in accordance with the invention is substantially monomeric as determined by SEC-MALLS.

In one embodiment, there is provided an isolated polypeptide or immunoglobulin domain in accordance with the invention wherein said isolated polypeptide or immunoglobulin has binding specificity for a target ligand. Suitably said isolated polypeptide or immunoglobulin displays antigen-binding activity. In one embodiment, the target ligand is a human antigen.

In another embodiment, there is provided an isolated polypeptide or immunoglobulin domain in accordance with any aspect or embodiment of the invention wherein said isolated polypeptide with framework mutations at least one of positions 36, 38, 43, 44, 46 or 87 has improved antigen-binding activity to human serum albumin when compared with the parent molecule as a result of decreased dissociation equilibrium constant K_{D}.

In another aspect, the invention provides a list of polypeptides comprising the polypeptides or immunoglobulins in accordance with the invention wherein at least 60, 70, 75, 80, 85, or 90% of the polypeptides are in monomeric form as determined by SEC-MALLS or AUC (see experimental section).

A further aspect provides a library comprising a polypeptide or variant immunoglobulin light chain variable domain regions in accordance with the invention wherein at least one of amino acid positions 36, 38, 43, 44, 46 or 87 has been mutated, said positions being assigned in accordance with the Kabat amino acid numbering system.

A further aspect which may be mentioned provides a library comprising a polypeptide or variant immunoglobulin light chain variable domain regions in accordance with the invention wherein at least one of amino acid positions 38, 43 and 44 has been mutated, said positions being assigned in accordance with the Kabat amino acid numbering system.

Yet another aspect of the invention provides a library of Vκ immunoglobulin domains wherein position 43 is selected from D, I, L, K or E.

Yet another aspect of the invention provides a library of Vκ immunoglobulin domains wherein position 46 is selected from R, D, Q, E or F, such as D.

Yet another aspect of the invention provides a library of Vκ immunoglobulin domains wherein position 87 is selected from D, C, L or F, such as L.

In one embodiment, the library is a V_{κ} DPK9 library.

Another aspect provides a library for expressing polypeptides or variant immunoglobulin light chain variable domain regions in accordance with the invention comprising a list of nucleic acid sequences encoding said polypeptides or immunoglobulin light chain variable domains.

There is also provided a library of nucleic acids encoding a polypeptide or a immunoglobulin light chain single variable domain in accordance with the invention.

In one aspect, the invention provides a list or a library in accordance with the invention wherein said library further comprises diversity in the CDR regions. Diversity in CDR regions can be generated by suitable methods.

Another aspect provides a nucleic acid encoding a polypeptide or immunoglobulin light chain single variable domain in accordance with the invention.

The invention provides a pharmaceutical composition comprising a polypeptide or an immunoglobulin single variable domain in accordance with the invention as well as a polypeptide or immunoglobulin single variable domain in accordance with the invention for use as a medicament. Said pharmaceutical composition may be suitable for different forms of administration familiar to those skilled in the art and may comprise pharmaceutically acceptable carriers or excipients. Furthermore, the invention provides a method of treatment comprising administering a polypeptide or immunoglobulin single variable domain in accordance with the invention to a person in need of treatment.

A polypeptide or immunoglobulin light chain single variable domain in accordance with the invention may be part of a larger fusion protein or bi- or multi-specific molecule. Suitable larger constructs include dAb-dAb, mAb-dAb or dAb-polypeptide constructs.

The invention further provides a process for making a dAb comprising introducing mutations in accordance with the invention.

### BRIEF DESCRIPTION OF THE DRAWINGS

FIG. 1: Sensorgram traces for 2.5 μM dAbs binding to Protein L. SM=stable monomer, SD=stable dimer, RE=rapid equilibrium between monomer and dimer. Resp 1=response point 1, Resp 2=response point 2.

FIG. 2: Sensorgram traces (RU—vertical axis; time (s)—horizontal axis) for 31.25 nM dAbs binding to Protein L. DOM7h-8 parent molecule is a dimeric Vk dAb and DOM7h-8 P44Q is a monomeric Vk dAb.

FIG. 3: Graph summarising supernatant Protein L binding data. Horizontal bars indicate the mean.

### DETAILED DESCRIPTION OF THE INVENTION

Within this specification the invention has been described, with reference to embodiments, in a way which enables a clear and concise specification to be written. It is intended and should be appreciated that embodiments may be variously combined or separated without parting from the invention.

Unless defined otherwise, all technical and scientific terms used herein have the same meaning as commonly understood by one of ordinary skill in the art (e.g, in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc. which are incorporated herein by reference) and chemical methods.

As used herein, “immunoglobulin” refers to a family of polypeptides which retain the immunoglobulin fold characteristic of antibody molecules, which contain two β sheets and, usually, a conserved disulphide bond. Members of the immunoglobulin superfamily are involved in many aspects of cellular and non-cellular interactions in vivo, including widespread roles in the immune system (for example, antibodies, T-cell receptor molecules and the like), involvement in cell adhesion (for example the ICAM molecules) and intracellular signaling (for example, receptor molecules, such as the PDGF receptor). The present invention is applicable to all immunoglobulin superfamily molecules which possess binding domains. In one embodiment, the present invention relates to antibodies.

As used herein “domain” refers to a folded protein structure which retains its tertiary structure independently of the rest of the protein. Generally, domains are responsible for discrete functional properties of proteins and in many cases may be added, removed or transferred to other proteins without loss of function of the remainder of the protein and/or of the domain. By single antibody variable domain or immunoglobulin single variable domain is meant a folded polypeptide domain comprising sequences characteristic of antibody variable domains. It therefore includes complete antibody variable domains and modified variable domains, for example in which one or more loops have been replaced by sequences which are not characteristic of antibody variable domains, or antibody variable domains which have been truncated or comprise N- or C-terminal extensions, as well as folded fragments of variable domains which retain at least in part the binding activity and specificity of the full-length domain.

A “V_{K} DPK9 immunoglobulin domain” (also written as “DPₖ9”) is an immunoglobulin domain derived from the human framework O12/O2/DPK9. Such a domain may further comprise sequences derived from the human framework Jk1. Immunoglobulin domains may be derived from other human framework regions. An analysis of the structural repertoire of the human Vκ domain is described, for example, in Tomlinson et al. (1995), EMBO 14; p. 1628-38. In addition, the structural differences between the repertoires of mouse and human germline, genes is described, for example, in Amalgro et al. (1998); Immunogenetics; 47; p. 355-363.

The phrase “immunoglobulin single variable domain” refers to an antibody variable domain (V_{H}, V_{HH}, V_{L}) or binding domain that specifically binds an antigen or epitope independently of different or other V regions or domains. An immunoglobulin single variable domain can be present in a format (e.g, homo- or hetero-multimer) with other variable regions or variable domains where the other regions or domains are not required for antigen binding by the single immunoglobulin variable domain (i.e., where the immunoglobulin single variable domain binds antigen independently of the additional variable domains). A “domain antibody” or “dAb” is an “immunoglobulin single variable domain” as the term is used herein. A “single antibody variable domain” or an “antibody single variable domain” is the same as an “immunoglobulin single variable domain” as the term is used herein. An immunoglobulin single variable domain is in one embodiment a human antibody variable domain, but also includes single antibody variable domains from other species such as rodent (for example, as disclosed in WO 00/29004, the contents of which are incorporated herein by reference in their entirety), nurse shark and Camelid V_{H}H dAbs. Camelid V_{H}H are immunoglobulin single variable domain polypeptides that are derived from species including camel, llama, alpaca, dromedary, and guanaco, which produce heavy chain antibodies naturally devoid of light chains. The V_{H}H may be humanized.

In all aspects of the invention, the or each immunoglobulin single variable domain is independently selected from antibody heavy chain and light chain single variable domains, e.g. V_{H}, V_{L} and V_{H}H. Antibody heavy chain domains are indicated by VH or V_{H}, VHH, V_{H}H or V_{HH}. Antibody light chain domains are indicated by VL or V_{L}. A “variant” with reference to an immunoglobulin light chain single variable domain is one which comprises the amino acid sequence of a naturally occurring, germ line or parental immunoglobulin light chain but differs in one or more amino acids. That is a “variant” comprises one or more amino acid differences when compared to a naturally occurring sequence or “parental” sequence from which it is derived. Suitably a “parental” sequence is a naturally occurring immunoglobulin light chain single variable domain sequence, a germ line immunoglobulin light chain sequence or an amino acid sequence of an immunoglobulin light chain single variable domain which has been identified to bind to an antigen of interest. In one embodiment, the parental sequence may be selected from a library such as a 4 G or 6 G library described in WO2005093074 and WO04101790, respectively.

A “lineage” refers to a series of immunoglobulin single variable domains that are derived from the same “parental” clone. For example, a lineage comprising a number of variant clones may be generated from a parental or starting immunoglobulin single variable domain by diversification, site directed mutagenesis, generation of error prone or doped libraries. Suitably binding molecules are generated in a process of affinity maturation. Suitable assays and screening methods for identifying an immunoglobulin light chain single variable domain are described, for example in PCT/EP2010/052008 and PCT/EP2010/052007, for example. A “parental” sequence includes immunoglobulin single variable domains such as DOM7h-8 as described herein. Suitably, said variants may also include variation in the CDR sequences, such variation contributing to differences in antigen specificity.

In one embodiment, the parental sequence may be modified in accordance with the invention so as to improve one or more of the biophysical properties, including solution state (measured, for example by MALLS and/or SEC MALLS or AUC) and thermostability (measured, for example, by DSC). In one embodiment, the variant has an amino acid substitution at one or more amino acid positions within the immunoglobulin light chain single variable domain. Immunoglobulin light chain single variable domains in accordance with the invention can form monomers, dimers, trimers or multimers in solution. The different oligomers may be in equilibrium with each other. Equilibrium may be fast or slow. By “substantially monomeric” it is meant that the predominant form of the single variable domain is monomeric in solution. Solution state can be measured by SEC-MALLS as described herein or AUC. Suitably, the invention provides a (substantially) pure monomer. In one embodiment, the dAb is at least 70, 75, 80, 85, 90, 95, 98, 99, 99.5% pure or 100% pure monomer. Similarly by “substantially dimeric” it is meant that the predominant form in solution is a dimeric form. In one embodiment, a dimeric form of a dAb is at least 70, 75, 80, 85, 90, 95, 98, 99, 99.5% pure or 100% pure dimer. Suitably where monomeric/dimeric state is measured by SEC MALLS, the dAb concentration may be in the range of 5 to 10 μM.

In one embodiment, the immunoglobulin single variable domain, polypeptide or ligand in accordance with the invention can be provided in any antibody format. As used herein, “antibody format” refers to any suitable polypeptide structure in which one or more antibody variable domains can be incorporated so as to confer binding specificity for antigen on the structure. A variety of suitable antibody formats are known in the art, such as, chimeric antibodies, humanized antibodies, human antibodies, single chain antibodies, bispecific antibodies, antibody heavy chains, antibody light chains, homodimers and heterodimers of antibody heavy chains and/or light chains, antigen-binding fragments of any of the foregoing (e.g, a Fv fragment (e.g, single chain Fv (scFv), a disulfide bonded Fv), a Fab fragment, a Fab′ fragment, a F(ab′)₂ fragment), a single antibody variable domain (e.g, a dAb, V_{H}, V_{HH}, V_{L}), and modified versions of any of the foregoing (e.g, modified by the covalent attachment of polyethylene glycol or other suitable polymer or a humanized V_{HH}).

As used herein an “antibody” refers to IgG, IgM, IgA, IgD or IgE or a fragment (such as a Fab, F(ab′)₂, Fv, disulphide linked Fv, scFv, closed conformation multispecific antibody, disulphide-linked scFv, diabody) whether derived from any species naturally producing an antibody, or created by recombinant DNA technology; whether isolated from, for example, serum, B-cells, hybridomas, transfectomas, yeast or bacteria.

As described herein an “antigen” is a molecule that is bound by a binding domain according to the present invention. Typically, antigens are bound by antibody ligands and are capable of raising an antibody response in vivo. It may be, for example, a polypeptide, protein, nucleic acid or other molecule.

As used herein, the phrase “target” refers to a biological molecule (e.g, peptide, polypeptide, protein, lipid, carbohydrate) to which a polypeptide domain which has a binding site can bind. The target can be, for example, an intracellular target (e.g, an intracellular protein target), a soluble target (e.g, a secreted), or a cell surface target (e.g, a membrane protein, a receptor protein). Suitably a target is a molecule having a role in a disease such that binding said target with a binding molecule in accordance with the invention may play a role in amelioration or treatment of said disease. The target antigen may be, or be part of, polypeptides, proteins or nucleic acids, which may be naturally occurring or synthetic. In this respect, the ligand of the invention may bind the target antigen and act as an antagonist or agonist (e.g., EPO receptor agonist). One skilled in the art will appreciate that the choice is large and varied. They may be for instance, human or animal proteins, cytokines, cytokine receptors, where cytokine receptors include receptors for cytokines, enzymes, co-factors for enzymes or DNA binding proteins.

In one embodiment, the immunoglobulin single variable domain or polypeptide in accordance with the invention can be part of a “dual-specific ligand” which refers to a ligand comprising a first antigen or epitope binding site (e.g., first immunoglobulin single variable domain) and a second antigen or epitope binding site (e.g., second immunoglobulin single variable domain), wherein the binding sites or variable domains are capable of binding to two antigens (e.g., different antigens or two copies of the same antigen) or two epitopes on the same antigen which are not normally bound by a monospecific immunoglobulin. For example, the two epitopes may be on the same antigen, but are not the same epitope or sufficiently adjacent to be bound by a monospecific ligand. In one embodiment, dual specific ligands according to the invention are composed of binding sites or variable domains which have different specificities, and do not contain mutually complementary variable domain pairs (i.e. V_{H}/V_{L} pairs) which have the same specificity (i.e., do not form a unitary binding site).

Dual-specific ligands and suitable methods for preparing dual-specific ligands are disclosed in WO 2004/058821, WO 2004/003019, and WO 03/002609, the entire teachings of each of these published international applications are incorporated herein by reference.

In one embodiment, immunoglobulin single variable domains in accordance with the invention may be used to generate dual or multi-specific compositions or fusion polypeptides. Accordingly, immunoglobulin single variable domains in accordance with the invention may be used in larger constructs. Suitable constructs include fusion proteins between an anti-SA immunoglobulin single variable domain (dAb) and a monoclonal antibody, NCE, protein or polypeptide and so forth. Accordingly, anti-SA immunoglobulin single variable domains in accordance with the invention may be used to construct multi-specific molecules, for example, bi-specific molecules such as dAb-dAb (i.e. two linked immunoglobulin single variable domains in which one is an anti-SA dAb), mAb-dAb or polypeptide-dAb constructs. In these constructs the anti-SA dAb (AlbudAb™) component provides for half-life extension through binding to serum albumin (SA). Suitable mAb-dAbs and methods for generating these constructs are described, for example, in WO2009/068649.

In addition, WO04003019 and WO2008/096158 disclose anti-serum albumin (SA) binding moieties, such as anti-SA immunoglobulin single variable domains (dAbs), which have therapeutically-useful half-lives. These documents disclose monomer anti-SA dAbs as well as multi-specific ligands comprising such dAbs, e.g., ligands comprising an anti-SA dAb and a dAb that specifically binds a target antigen, such as TNFR1. Binding moieties are disclosed that specifically bind serum albumins from more than one species, e.g. human/mouse cross-reactive anti-SA dAbs.

WO05118642 and WO2006/059106 disclose the concept of conjugating or associating an anti-SA binding moiety, such as an anti-SA immunoglobulin single variable domain, to a drug, in order to increase the half-life of the drug. Protein, peptide and new chemical entity (NCE) drugs are disclosed and exemplified. WO2006/059106 discloses the use of this concept to increase the half-life of insulintropic agents, e.g., incretin hormones such as glucagon-like peptide (GLP)-1.

Reference is also made to Holt et al, “Anti-Serum albumin domain antibodies for extending the half-lives of short lived drugs”, Protein Engineering, Design & Selection, vol 21, no 5, pp 283-288, 2008.

The invention also provides canonical structures of the claimed polypeptides. Analysis of the structures and sequences of domain antibodies (dAbs) has shown that six antigen binding loops (3 from the VH domain and 3 from the Vκ domain) have a small repertoire of main chain conformations, or canonical structures (Chothia C & Lesk A M. (1987). Canonical structures for the hypervariable regions of immunoglobulins. J Mol Biol. 196, 901-17; Chothia et al. (1989). Conformations of immunoglobulin hypervariable regions. Nature, 342, 877-883; Tomlinson et al. (1995) supra).

The canonical structures are determined by

1. the length of the antigen binding loop;

2. specific residues at key sites in the loop itself and in the antibody framework. Canonical structures of the human Vκ domains are described by Tomlinson et al., (1995). References herein to Vκ domains are based on the single framework comprising κ light chain genes O12/O2/DPK9 and JK1 with side chain diversity incorporated at positions in the antigen binding site. The canonical structure of the Vκ domain encoded by this framework is 2:1:1 (Tomlinson et al., 1995). The key structural residues for canonical structures of each of the three loops (L1, L2, L3) are generally not diversified to preserve these main chain conformations.

The invention also provides isolated and/or recombinant nucleic acid molecules encoding ligands (single variable domains, fusion proteins, polypeptides, dual-specific ligands and multispecific ligands) as described herein.

The invention also provides a vector comprising a recombinant nucleic acid molecule of the invention. In certain embodiments, the vector is an expression vector comprising one or more expression control elements or sequences that are operably linked to the recombinant nucleic acid of the invention. The invention also provides a recombinant host cell comprising a recombinant nucleic acid molecule or vector of the invention. Suitable vectors (e.g, plasmids, phagemids), expression control elements, host cells and methods for producing recombinant host cells of the invention are well-known in the art, and examples are further described herein.

Suitable expression vectors can contain a number of components, for example, an origin of replication, a selectable marker gene, one or more expression control elements, such as a transcription control element (e.g, promoter, enhancer, terminator) and/or one or more translation signals, a signal sequence or leader sequence, and the like. Expression control elements and a signal sequence, if present, can be provided by the vector or other source. For example, the transcriptional and/or translational control sequences of a cloned nucleic acid encoding an antibody chain can be used to direct expression.

A promoter can be provided for expression in a desired host cell. Promoters can be constitutive or inducible. For example, a promoter can be operably linked to a nucleic acid encoding an antibody, antibody chain or portion thereof, such that it directs transcription of the nucleic acid. A variety of suitable promoters for prokaryotic (e.g, lac, tac, T3, T7 promoters for E. coli) and eukaryotic (e.g, Simian Virus 40 early or late promoter, Rous sarcoma virus long terminal repeat promoter, cytomegalovirus promoter, adenovirus late promoter) hosts are available.

In addition, expression vectors typically comprise a selectable marker for selection of host cells carrying the vector, and, in the case of a replicable expression vector, an origin of replication. Genes encoding products which confer antibiotic or drug resistance are common selectable markers and may be used in prokaryotic (e.g., lactamase gene (ampicillin resistance), Tet gene for tetracycline resistance) and eukaryotic cells (e.g, neomycin (G418 or geneticin), gpt (mycophenolic acid), ampicillin, or hygromycin resistance genes). Dihydrofolate reductase marker genes permit selection with methotrexate in a variety of hosts. Genes encoding the gene product of auxotrophic markers of the host (e.g, LEU2, URA3, HIS3) are often used as selectable markers in yeast. Use of viral (e.g, baculovirus) or phage vectors, and vectors which are capable of integrating into the genome of the host cell, such as retroviral vectors, are also contemplated. Suitable expression vectors for expression in mammalian cells and prokaryotic cells (E. coli), insect cells (Drosophila Schnieder S2 cells, Sf9) and yeast (P. methanolica, P. pastoris, S. cerevisiae) are well-known in the art.

Suitable host cells can be prokaryotic, including bacterial cells such as E. coli, B. subtilis and/or other suitable bacteria; eukaryotic cells, such as fungal or yeast cells (e.g., Pichia pastoris, Aspergillus sp., Saccharomyces cerevisiae, Schizosaccharomyces pombe, Neurospora crassa), or other lower eukaryotic cells, and cells of higher eukaryotes such as those from insects (e.g., Drosophila Schnieder S2 cells, Sf9 insect cells (WO 94/26087 (O'Connor)), mammals (e.g., COS cells, such as COS-1 (ATCC Accession No. CRL-1650) and COS-7 (ATCC Accession No. CRL-1651), CHO (e.g., ATCC Accession No. CRL-9096, CHO DG44 (Urlaub, G. and Chasin, L A., Proc. Natl. Acad. Sci. USA, 77(7):4216-4220 (1980))), 293 (ATCC Accession No. CRL-1573), HeLa (ATCC Accession No. CCL-2), CV1 (ATCC Accession No. CCL-70), WOP (Dailey, L., et al., J. Virol., 54:739-749 (1985), 3T3, 293T (Pear, W. S., et al., Proc. Natl. Acad. Sci. U.S.A., 90:8392-8396 (1993)) NS0 cells, SP2/0, HuT 78 cells and the like, or plants (e.g., tobacco). (See, for example, Ausubel, F. M. et al., eds. Current Protocols in Molecular Biology, Greene Publishing Associates and John Wiley & Sons Inc. (1993).) In some embodiments, the host cell is an isolated host cell and is not part of a multicellular organism (e.g., plant or animal). In certain embodiments, the host cell is a non-human host cell.

In one embodiment, the polypeptides or immunoglobulin single variable domains in accordance with the invention are secreted when expressed in a suitable expression system. Suitably, the amino acid replacements or mutations in accordance with the invention do not lead to loss of expression.

Additional expression systems include cell free systems such as those described in In yet another embodiment, expression of variable domains can be accomplished using cell-free expression systems such as those described in PCT/GB2005/003243 and WO2006/046042.

Reference is made to WO200708515, page 161, line 24 to page 189, line 10 for details of disclosure that is applicable to embodiments of the present invention. This disclosure is hereby incorporated herein by reference as though it appears explicitly in the text of the present disclosure and relates to the embodiments of the present invention, and to provide explicit support for disclosure to incorporate into claims below. This includes disclosure presented in WO200708515, page 161, line 24 to page 189, line 10 providing details of the “Preparation of Immunoglobulin Based Ligands”, “Library vector systems”, “Library Construction”, “Combining Single Variable Domains”, “Characterisation of Ligands”, “Therapeutic and diagnostic compositions and uses”, as well as definitions of “operably linked”, “naive”, “prevention”, “suppression”, “treatment”, “therapeutically-effective dose” and “effective”.

### EXAMPLES

### Methods

SEC and SEC MALLS (size exclusion chromatography with multi-angle-LASER-light-scattering) is a non-invasive technique for the characterisation of macromolecules in solution. Briefly, proteins (routinely at concentration of 1 mg/ml in buffer Dulbecco's PBS) are separated according to their hydrodynamic properties by size exclusion chromatography (Columns used are: Tosoh Biosciences TSK gel3000 G3000SWXL and Superdex200 or 75 10/300GL, respectively (cat #: 17-5175-01 and 17-5174-01)) in PBS.

Following separation, the propensity of the protein to scatter light is measured using a multi-angle-LASER-light-scattering (MALLS) detector (Wyatt, US). The intensity of the scattered light while protein passes through the detector is measured as a function of angle. This measurement taken together with the protein concentration determined using the refractive index (RI) detector allows calculation of the molar mass using appropriate equations (integral part of the analysis software Astra v.5.3.4.12). The highest concentration at the mid-point of the eluting peak is about 8-10 μM and this consequently is the concentration at which MALLS determines the in-solution (monomer/dimer) state of the protein.

Differential scanning calorimetry (DSC) is a thermoanalytical technique in which the difference in the amount of heat required to increase the temperature of a sample and reference are measured as a function of temperature. It can be used to study a wide range of thermal transitions in proteins and is useful for determining the melting temperatures as well as thermodynamic parameters. Briefly, the protein is heated at a constant rate of 180 degrees C./hr (at 1 mg/ml routinely in PBS) and a detectable heat capacity change associated with thermal denaturation is measured as a function of temperature. The transition midpoint (Tₘ) is determined, which is described as the temperature where 50% of the protein is in its native conformation and the other 50% is denatured. Here, DSC determined the apparent transition midpoint (ₐₚₚTₘ) as most of the proteins examined do not unfold fully reversibly. The higher the Tm or appTm, the more stable the molecule. In the present examples, the software package used was Origin® v7.0383 (OriginLab).

Analytical Ultra-Centrifugation (AUC): Sedimentation equilibrium is a method for measuring solution molecular mass (described, for example, in Lebowitz et al. Protein Science (2002), 11:2067-2079).

In the present examples, three 6-channel equilibrium cells were loaded with 9 protein solutions made by diluting the stock sample 10-, 20-, 30-, 150-, 200-, 300-, 400-, 500, and 600-fold (a range from 540 to 90 μg/ml). Each sample channel was loaded with 120 μl of protein solution and the reference channels were loaded with 125 μl of Dulbecco's phosphate-buffered saline (DPBS) dilution buffer. These cells were then loaded into an AN90-TI rotor and placed into a Beckman Coulter ProteomeLab XL-1 analytical centrifuge equipped with both absorbance and Rayleigh interference (refractive index detection) optical systems. Absorbance scans for the three highest concentrations were recorded at 280 nm; for the lowest concentrations 230 nm was used. The temperature was set at 25° C.

The rotor was then brought to 25,000 rpm. The cells were then scanned after 12, 16, and 20 hr at 25,000 rpm. At the end of the run the rotor speed was increased to 48,000 rpm and a single ‘overspeed’ scan was recorded 8 hr later in order to experimentally measure the baseline offsets.

The resulting data were analysed using the KDALTON program (Alliance Protein Laboratories, Philo et al. (1994), J. Biol. Chem., 269, p. 27840-27846; Philo, J. S. (2000), Methods Enzymol. 321, 100-120). A polypeptide partial specific volume of 0.7256 ml/g at 25° C. was calculated based on the theoretical amino acid composition (calculated from the supplied amino acid sequence) using the program SEDNTERP (Laue et al. (1992) In: Analytical ultracentrifugation in biochemistry and polymer science. S. E. Harding, A. J. Rowe, and J. C. Horton, eds, Royal Society of Chemistry, pp. 90-125). The solvent density for DPBS at 25° C. was assigned as 1.03994 g/ml on measurements made previously.

Biacore Analysis: Surface Plasmon Resonance (SPR) (BIAcore™, GE Healthcare) experiments allow for the determination of binding kinetics and K_{D} of a ligand (dAb) to its antigen (e.g. serum albumin, Protein L etc.).

To determine the binding affinity (K_{D}) of a single albumin-binding dAb (AlbudAb™) to its antigen, purified dAbs were injected at a flow rate of 40 μl/min over human serum albumin (immobilised by primary-amine coupling onto CM5 chips; BIAcore) using AlbudAb concentrations from 5000 nM to 39 nM (5000 nM, 2500 nM, 1250 nM, 625 nM, 312 nM, 156 nM, 78 nM, 39 nM) in HBS-EP BIAcore buffer. The data analysis followed routine and established algorithms using the instrument's software (Bia-evaluation 3.2 RC1). The data analysis yields the following parameters:

K_{D}—[M]

kₐ—[M−1*sec−1]

k_{d}—[sec−1]

where K_{D} is dissociation equilibrium constant, M is molar concentration, kₐ is association rate constant, k_{d} is dissociation rate constant and sec is time.

Use of Protein L binding kinetics to predict dAb solution state: Protein L (also referred to as PpL) is a B-cell superantigen which was first discovered in the cell wall of Peptostreptococcus magnus (Björck L. (1998) Protein L. A novel bacterial cell wall protein with affinity for Ig L chains. J Immunol, 15; 140(4):1194-7) and binds immunoglobulin (Ig) light chain variable domains of the kappa isotype (Vκ) by interaction with residues in the framework 1 region (M. Graille, E. Stura, N. Housden, J. Beckingham, S. Bottomley, D. Beale, M. Taussig, B. Sutton, M. Gore, J. Charbonnier (2001) Complex between Peptostreptococcus magnus Protein L and a Human Antibody Reveals Structural Convergence in the Interaction Modes of Fab Binding Proteins. Structure, Volume 9, Issue 8, Pages 679-687). Depending on the strain, Protein L comprises either four (P. magnus strain 312) or five (P. magnus strain 3316), homologous (>70% protein sequence identity), tandem Vκ-binding domains, separated by flexible peptide linker regions (Kastern W, Sjöbring U, Björck L. (1992) Structure of peptostreptococcal protein L and identification of a repeated immunoglobulin light chain-binding domain. J Biol Chem., 25; 267(18):12820-5.). A strong avidity effect is observed when Protein L binds IgG or Fab molecules containing certain Vκ domains, which is presumed to be mediated by both the presence of multiple Protein L domains and the existence of high and low affinity binding interfaces within a single Protein L domain (Kastern et al., 1992).

It was postulated that a modulation of these avidity effects would be observed that could be correlated with the solution state of the dAb in question—i.e. monomers, dimers and other oligomerisation states would display differential binding kinetics to Protein L, under the correct conditions. In this manner, Protein L binding kinetics could be used as a surrogate for determining the solution state of a dAb. Real time kinetic Protein L:dAb binding data were therefore obtained by surface plasmon resonance (BIAcore) for a panel of dAbs with representative solution states.

Four-domain Protein L (derived from P. magnus₃₃₁₆; Sigma, P3101) and biotinylated Protein A (also referred to as b-PpA; Sigma P2165) were diluted to 10 μg/ml in pH 4.5 acetate buffer (BIAcore) and immobilised on a BIAcore CM5 chip. This resulted in a chip bearing the following: Fc1=blank, Fc2=363 RU b-PpA and Fc3=311 RU Protein L. A low surface density of Protein L and high flow rate were used in order to minimise rebinding of dAb to the chip surface.

A panel of eight purified Vκ dAbs with known representative solution states (determined previously by SEC-MALLS) were diluted to 2.5 μM in HBS-EP and then across 5 2-fold serial dilutions, down to 156 nM. Binding was measured by injection of 100 μl of each dilution at a flow rate of 50 μl/min and allowing 600 s of dissociation time on a BIAcore 3000 instrument (BIAcore, Sweden). The chip surface was regenerated between cycles with a 25 μl pulse of pH 2.5 Glycine buffer (BIAcore). Data from Fc3-2 was used for analysis.

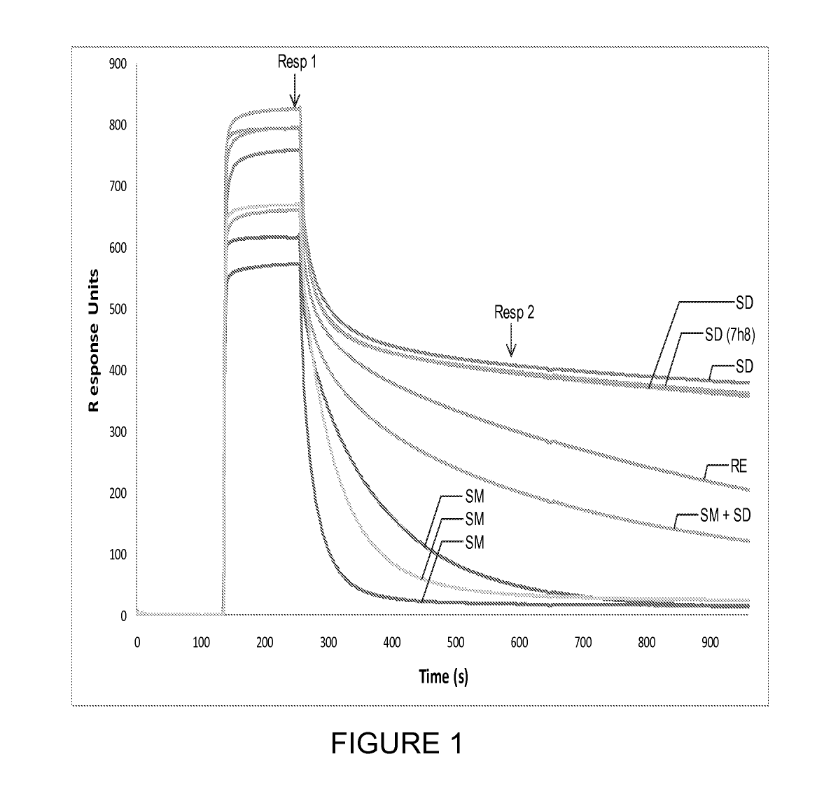

Representative sensorgram data is shown for the Protein L binding analysis of dAbs at 2.5 μM (FIG. 1). The position and shape of the sensorgrams shown were maintained across the concentration range for each dAb tested.

Following injection of the dAb across the chip derivatised with Protein L, report points placed at the end of the association phase (Response point 1, see FIG. 1) and 5 min into the dissociation phase (Response point 2, see FIG. 1), can be used to obtain the amount of dAb bound to Protein L at these time points (values from the relevant control flow cell are subtracted from these data). Using the following equation, it is possible to determine the proportion of dAb bound at 5 min (also referred to as % B₅): Resp 1/Resp2=% B₅.

If the dAb in question is monomeric, the % B₅ will be low (typically 0-5), but if the dAb in question is a dimer, the % B₅ will be high (typically 60-100). If the dAb sample in question exists in equilibrium between monomeric and dimeric solution states, or is composed of a mixture of monomers and dimers, the % B5 value will fall between that of monomeric or dimeric dAbs. The % B₅ value is therefore a numeric expression of the likely solution state of the dAb in question.

A clear difference was shown in Protein L binding kinetics for Vκ monomers and dimers, enabling differentiation between solution states, based both on the rate and extent of dissociation. Note that the relative position and shape of the curves for each dAb was constant, irrespective of the concentration analysed. Curve-fitting to a Langmuir 1:1 model was not attempted for the on-rate as this was judged to be too rapid, while fitting for off-rate (k_{d}) was precluded by the heavily biphasic nature of the dissociation curves.

Using the relevant control dAbs, it is possible to define the range between which monomers and dimers are found and thus predict the solution state of a dAb.

### Example 1

### Effect of A43D Mutation in Different V_{L} Immunoglobulin Single Variable Domains

A number of dAbs with binding affinities to antigens were taken and mutations introduced to replace amino acid at position 43 (A) with D. Mutations were introduced using site directed mutagenesis.

The following dAbs were taken:

PEP1-5-19 (anti-TNFalpha dAb):
(SEQ ID NO: 1)
DIQMTQSPSSLSASVGDRVTITCRASQSIDSYLHWYQQKPGKAPKLLIYS
ASELQSGVPSRFSGSGSGTDFTLTISSLQPEDFATYYCQQVVWRPFTFGQ
GTKVEIKR
DOM15-10 (anti-human VEGF dAb)
(SEQ ID NO: 2)
DIQMTQSPSSLSASVGDRVTITCRASQWIGPELSWYQQKPGKAPKLLIYH
TSILQSGVPSRFSGSGSGTDFTLTISSLQPEDFATYYCQQYMFQPRTFGQ
GTKVEIRR
DOM13-25-3 (anti-CEA dAb)
(SEQ ID NO: 3)
DIQMTQSPSSLSASVGDRVTITCRASQSIGPWLSWYQQKPGKAPKLLFYQ
VSRLQSGVPSRFSGSGSGTDFTLTIISLQPEDFATYYCQQNLAPPYTFGQ
GTKVEIKR
DOM9-155-25 (anti-IL-4 anti Fcn dAb)
(SEQ ID NO: 4)
DIQMTQSPSSLSASVGDRVTITCRASRPISDWLHWYQQKPGKAPKLLIAW
ASTLDSGVPSRFSGSGSGTDFTLTISSLQPEDFATYYCLQEGWGPPTFGQ
GTKVEIKR
DOM7h-14 (anti-HSA dAb)
(SEQ ID NO: 5)
DIQMTQSPSSLSASVGDRVTITCRASQWIGSQLSWYQQKPGKAPKLLIMW
RSSLQSGVPSRFSGSGSGTDFTLTISSLQPEDFATYYCAQGAALPRTFGQ
GTKVEIKR

In solution state was measured by SEC-MALLS as described above:

TABLE 1
Biophysical properties of dAbs and A43D mutants

| | | in-solution state with |
|---|---|---|
| | in-solution state WT | A43D |
| PEP1-5-19 | dimer | monomer |
| DOM15-10 | monomer | monomer |
| DOM13-25-3 | dimer | monomer |
| DOM9-155-25 | dimer | monomer |
| DOM7h-14 | monomer | monomer |

### Example 2

### Preparation and Analysis of DOM7h-8 or DOM7h-14 Libraries Mutagenised at Former Interface Residues

Background: Two Vκ dAbs derived from human light chain subgroups huVκI (DP_{K}9) were selected for mutation analysis, DOM7h-8 (described in WO05/118642) and DOM7h-14 (described in WO2008/096158), both of which bind Human Serum Albumin (HSA). For convenience, the DOM7h-8 clone used has a silent mutation that eliminates a BsaI restriction site (↓ indicates where the restriction enzyme cuts; the restriction enzyme recognition site is disrupted by a silent C to T mutation at position 51). Human Vκ light chains bind to Protein L (described in more detail below). Maintenance of Protein L binding gives a good indication of proper folding of an immunoglobulin domain.

The nucleotide and amino acid sequences of DOM7h-8 and DOM7h-14 used are given below:

DOM7h-8
Nucleotide-sequence:
(SEQ ID NO: 6)
GACATCCAGATGACCCAGTCTCCATCCTCCCTGTCTGCATC↓TGTAGGAG
ACCGTGTCACCATCACTTGCCGGGCAAGTCAGAGCATTAGCAGCTATTTA
AATTGGTATCAGCAGAAACCAGGGAAAGCCCCTAAGCTCCTGATCTATCG
GAATTCCCCTTTGCAAAGTGGGGTCCCATCACGTTTCAGTGGCAGTGGAT
CTGGGACAGATTTCACTCTCACCATCAGCAGTCTGCAACCTGAAGATTTT
GCTACGTACTACTGTCAACAGACGTATAGGGTGCCTCCTACGTTCGGCCA
AGGGACCAAGGTGGAAATCAAACGG
Amino acid-sequence:
(SEQ ID NO: 7)
DIQMTQSPSSLSASVGDRVTITCRASQSISSYLNWYQQKPGKAPKLLIYR
NSPLQSGVPSRFSGSGSGTDFTLTISSLQPEDFATYYCQQTYRVPPTFGQ
GTKVEIKR
DOM7h-14
Nucleotide-sequence:
(SEQ ID NO: 8)
GACATCCAGATGACCCAGTCTCCATCCTCCCTGTCTGCATCTGTAGGAGA
CCGTGTCACCATCACTTGCCGGGCAAGTCAGTGGATTGGGTCTCAGTTAT
CTTGGTACCAGCAGAAACCAGGGAAAGCCCCTAAGCTCCTGATCATGTGG
CGTTCCTCGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTGCTCAGGGTGCGGCGTTGCCTAGGACGTTCGGCCAA
GGGACCAAGGTGGAAATCAAACGG
Amino acid -sequence:
(SEQ ID NO: 9)
DIQMTQSPSSLSASVGDRVTITCRASQWIGSQLSWYQQKPGKAPKLLIMW
RSSLQSGVPSRFSGSGSGTDFTLTISSLQPEDFATYYCAQGAALPRTFGQ
GTKVEIKR

The biochemical properties of these dAbs are shown below.

TABLE 2
Biophysical properties and antigen-binding stoichiometry of
DOM7h-8 and DOM7h-14.

| | | | | Antigen- |
|---|---|---|---|---|
| | | | | Binding |
| | SEC- | | | Stoichi- |
| dAb | MALLS | DSCₐₚₚTₘ, ° C. | AUC data | ometry |
| DOM7h-8 | Dimer, | 69° C. | Dimer, (K_{D} for | 2:1 |
| | MW = | | self-association: | |
| | 23 kDa | | 120 nM in PBS) | |
| DOM7h-14 | Monomer | 2 Tms 60.6° C. | At high | 1:1 |
| | MW = | and 67.8° C.—for | concentrations | |
| | 12.5 kDa | dimer | the dAb | |
| | | dissociation and | dimerises (K_{D} | |
| | | monomer | for self | |
| | | denaturation | association: | |
| | | respectively | 250 μM in PBS) | |

DOM7h-8 binds HSA as a dimer (see Table 2). Residues at the former V_{H}/V_{L} interface were chosen for analysis. These mutations are located in the conserved framework regions of the V_{κ} domain antibodies, as opposed to hypervariable CDR regions that confer the antigen-binding activity to the dAb.

DOM7h-14 exists predominantly as a monomer at concentrations below 250 μM in PBS (see Table 2). The inclusion of DOM7h-14 allows the impact of the mutations on the antigen- and protein L-binding activity of a dAb that is already predominantly monomeric to be assessed.

### Example 2A

### DOM7h-8

For DOM7h-8, 3 individual libraries were made with mutations at former V_{H}/V_{L} interface residues, Q38, A43 and P44:

Mutations were introduced by site-directed-mutagenesis using DOM7h-8 in the E. coli expression vector pDOM5 as a template (pDOM5 is a pUC119-based expression vector under control of the LacZ promoter). Site directed mutagenesis was performed by PCR using 100 ng of plasmid DNA as template and complementary primers each containing the required mutation. Reactions were hot-started by the addition of 2.5 U of PfuTurbo polymerase (Stratagene) to a PCR mix [100 ng of plasmid template, primers (2 μM each), dNTPs (0.2 mM each), 1% (v/v) formamide in 1×PfuTurbo buffer (Stratagene)]. Reactions were thermocycled [94° C. for 2 min; 18 times (94° C. for 30 sec, 55° C. for 30 sec, and 68° C. for 20 min); 68° C. for 2 min; 10° C. hold]. PCR reactions were purified with a QIAquick PCR purification kit (Qiagen) and eluted in 50 μl of H₂O. Purified DNA was restriction digested for 1 h with DpnI (New England Biolabs) to remove the input plasmid template. Restricted DNA samples were ethanol precipitated and suspended in 5 μL of H₂O. Precipitated DNA was transformed into chemically competent E. coli cells which were plated onto 2×TY/Carbenicillin 0.1 mg/ml plates and incubated overnight at 37° C.

Primers were as follows:

Q38 (primers:
(SEQ ID NO: 10)
5′-GCAGCTATTTAAATTGGTATCAGNNKAAACCAGGGAAAGCCCC-3′;
(SEQ ID NO: 11))
5′-GGGGCTTTCCCTGGTTTMNNCTGATACCAATTTAAATAGCTGC-3′,
A43 (primers:
(SEQ ID NO: 12)
5′-CAGCAGAAACCAGGGAAANNKCCTAAGCTCCTGATCTATCGG-3′;
(SEQ ID NO: 13))
5′-CCGATAGATCAGGAGCTTAGGMNNTTTCCCTGGTTTCTGCTG-3′,
P44 (primers:
(SEQ ID NO: 14)
5′-CAGCAGAAACCAGGGAAAGCCNNKAAGCTCCTGATCTATCGGAATTC
CC-3′;
(SEQ ID NO: 15))
5′-GGGAATTCCGATAGATCAGGAGCTTMNNGGCTTTCCCTGGTTTCTGC
TG-3′,

The NNK codon used to introduce diversity encodes all 20 amino acids and the TAG stop codon. Clones identified as binding to Protein L were sequenced with primer DOM8 (AGCGGATAACAATTTCACACAGGA (SEQ ID NO: 16)).

96 Colonies were picked at random from each library into a 96 well plate format and expressed in 1 ml 2×TY 0.1 mg/ml carbenicillin supplemented with OnEx solutions 1, 2 and 3 according to the manufacturer's instructions (Novagen). Cultures were grown at 30° C. for 3 days at 950 rpm high humidity in an InforsHT shaker. Cells were pelleted by centrifugation (4.5 k rpm in bench top Sorvall centrifuge for 30 mins) and 75 μl of the supernatant added to an equal volume of HBS-EP buffer (GE Healthcare). Expressed supernatants were screened by BIAcore for Protein L binding using biotinylated Protein L (Pierce) coupled to a streptavidin coated BIAcore chip (495 RU). Clones identified as binding to Protein L were sequenced with primer DOM8 (SEQ ID NO: 16 as defined hereinbefore).

In order to obtain the full complement of amino acid variation at positions Q38, A43 and P44 clones not identified in the random screening of the library were made by site-directed-mutagenesis using DOM7h-8 in the E. coli expression vector pDOM5 as a template with primers listed in Table 3.

TABLE 3
Primer pairs used to generate DOM7h-8 mutants
not identified in the NNK libraries
(described above) at positions Q38, A43 or P44

| | |
|---|---|
| Q38C | GCAGCTATTTAAATTGGTATCAGTGCAAACCAGGGAAAGCCCC; |
| | (SEQ ID NO: 17) |
| | GGGGCTTTCCCTGGTTTGCACTGATACCAATTTAAATAGCTGC |
| | (SEQ ID NO: 18) |
| Q38 | GCAGCTATTTAAATTGGTATCAGAAAAAACCAGGGAAAGCCCC; |
| K | (SEQ ID NO: 19) |
| | GGGGCTTTCCCTGGTTTTTTCTGATACCAATTTAAATAGCTGC |
| | (SEQ ID NO: 20) |
| A43 | GGTATCAGCAGAAACCAGGGAAAAACCCTAAGCTCCTGATCTATCG |
| N | G; |
| | (SEQ ID NO: 21) |
| | CCGATAGATCAGGAGCTTAGGGTTTTTCCCTGGTTTCTGCTGATAC |
| | C |
| | (SEQ ID NO: 22) |
| A43 | CAGCAGAAACCAGGGAAAGATCCTAAGCTCCTGATCTATC |
| D | (SEQ ID NO: 23) |
| | GATAGATCAGGAGCTTAGGATCTTTCCCTGGTTTCTGCTG |
| | (SEQ ID NO: 24) |
| A43 | CGGTATCAGCAGAAACCAGGGAAATGCCCTAAGCTCCTGATCTATC |
| | GG |
| | (SEQ ID NO: 25) |
| | CCGATAGATCAGGAGCTTAGGGCATTTCCCTGGTTTCTGCTGATAC |
| | C |
| | (SEQ ID NO: 26) |
| A43I | GGTATCAGCAGAAACCAGGGAAAATTCCTAAGCTCCTGATCTATCG |
| | G |
| | (SEQ ID NO: 27) |
| | CCGATAGATCAGGAGCTTAGGAATTTTCCCTGGTTTCTGCTGATAC |
| | C |
| | (SEQ ID NO: 28) |
| P44C | CAGCAGAAACCAGGGAAAGCCTGCAAGCTCCTGATCTATCGGAATT |
| | CCC |
| | (SEQ ID NO: 29) |
| | GGGAATTCCGATAGATCAGGAGCTTGCAGGCTTTCCCTGGTTTCTG |
| | CTG |
| | (SEQ ID NO: 30) |
| P44E | CAGCAGAAACCAGGGAAAGCCGAAAAGCTCCTGATCTATCGGAATT |
| | CCC |
| | (SEQ ID NO: 31) |
| | CAGCAGAAACCAGGGAAAGCCGAAAAGCTCCTGATCTATCGGAATT |
| | CCC |
| | (SEQ ID NO: 32) |
| P44T | CAGCAGAAACCAGGGAAAGCCACCAAGCTCCTGATCTATCGGAATT |
| | CCC |
| | (SEQ ID NO: 33) |
| | GGGAATTCCGATAGATCAGGAGCTTGGTGGCTTTCCCTGGTTTCTG |
| | CTG |
| | (SEQ ID NO: 34) |
| P44 | CAGCAGAAACCAGGGAAAGCCTGGAAGCTCCTGATCTATCGGAATT |
| W | CCC |
| | (SEQ ID NO: 35) |
| | GGGAATTCCGATAGATCAGGAGCTTCCAGGCTTTCCCTGGTTTCTG |
| | CTG |
| | (SEQ ID NO: 36) |

Screening of DOM7h-8 mutants: DOM7h-8 mutants at positions Q38, A43 or P44 were screened by BIAcore both before and after purification from bacterial supernatant in order to characterize dAb binding activity to cognate HSA binding and superantigen Protein L. SEC and SEC MALLS on purified proteins were used to characterise the oligomerization state of the parent dAb and mutants.

Screening of dAbs in bacterial supernatants for Protein L and HSA-binding activity: Bacterial clones were picked into a 96 well plate format and expressed in 1 ml 2×TY 0.1 mg/ml carbenicillin supplemented with OnEx solutions 1, 2 and 3 according to the manufacturer's instructions (Novagen). Cultures were grown at 30° C. for 3 days at 950 rpm high humidity in an InforsHT shaker. Cells were pelleted by centrifugation (4.5 k in a bench top Sorvall centrifuge for 30 mins) and 75 μl of the supernatant added to an equal volume of HBS-EP buffer (GE Healthcare). Diluted supernatants were screened by BIAcore for Protein L binding using Protein L (Sigma) coupled to a CM5 BIAcore chip (789 RU) and HSA coupled on a separate flow cell on the same CM5 chip (6036 RU) (see Tables 4 to 6).

Purification of Vκ dAbs to assay for Protein L and HSA-binding and for SEC and SEC MALLS analysis: Protein from all clones expressing mutants of DOM7h-8 at positions Q38, A43 or P44 was expressed in 0.51 cultures in 2×TY Carbenicillin 100 μg/ml, antifoam, supplemented with OnEx solutions 1, 2 and 3 according to the manufacturer's instructions (Novagen). Cultures were grown at 30° C. for 3 days at 250 rpm in an InforsHT shaker at 250 rpm. Cultures were centrifuged at 4,500 rpm in a benchtop centrifuge for 45 min and protein purified from clarified supernatants by batch binding to 15 ml of Streamline Protein L for 2 h with rotation. After extensive washing with high salt PBS buffer protein was eluted from the resin at purities >95% with 0.1M Glycine pH 2. Prior to any further biochemical/-physical characterisation, the proteins were concentrated and buffer exchanged into PBS.

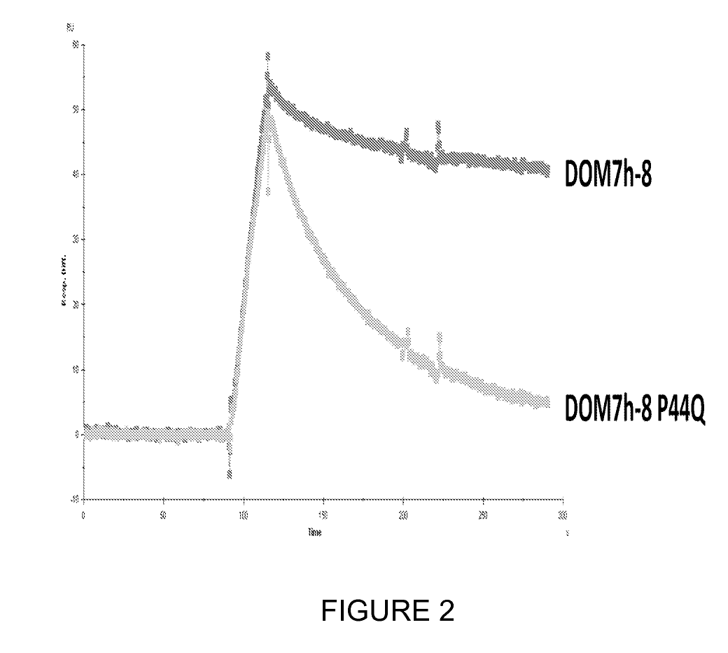

Purified proteins at concentrations ranging from 1 μM, 500 nM, 250 nM, 125 nM, 62.5 nM and 31.25 nM were assayed by BIAcore for binding to Protein L (311 RU) and binding to HSA (559 RU) coupled to separate flow cells on a CM5 chip. Those clones that dissociated from Protein L significantly faster than the parent molecule DOM7h-8 (a dimer) were assigned to be either stable monomers or monomers in equilibrium with dimers (see FIG. 2; Tables 4 to 6). Purified proteins were also analysed for HSA binding to assess the effect mutations have on the conformation of CDR regions of the dAb that make contact with antigen (see Tables 4 to 6).

Purified proteins at concentrations ranging from 0.5 mg/ml and 1.6 mg/ml were analysed by SEC and/or SEC MALLS to determine their in-solution state (see Tables 4 to 6).

TABLE 4

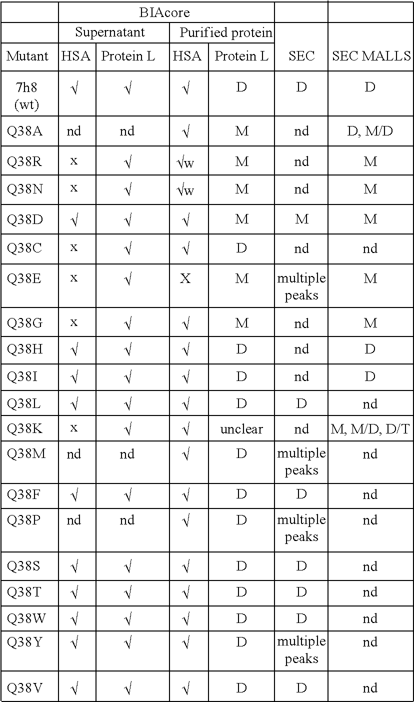

TABLE 5

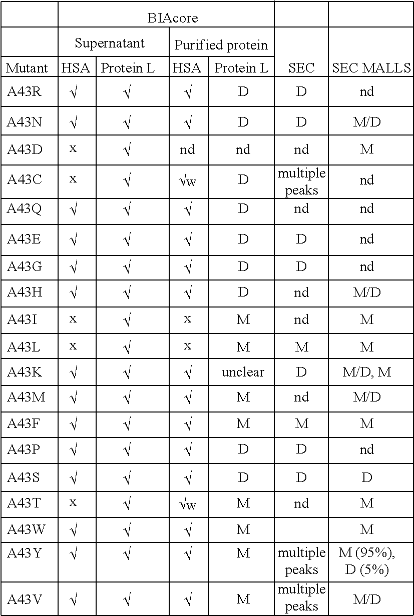

TABLE 6

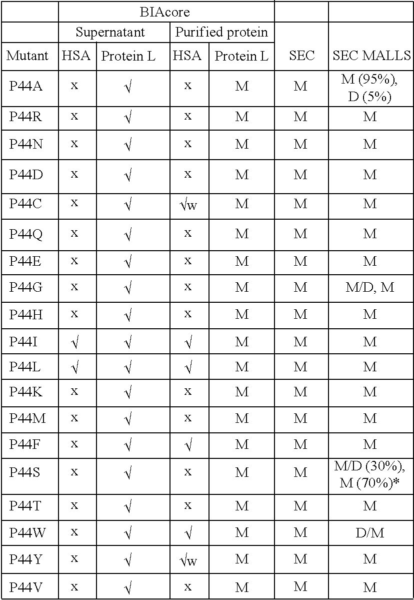

Tables 4-6: BIAcore and biophysical analysis of DOM7h-8 expressed supernatants and purified protein. The shaded rows identify mutations that monomerise DOM7h-8 Vκ dAb dimer. (x—indicates no binding to immobilized ligand on BIAcore chip; √—indicates good binding to immobilized ligand on BIAcorc chip; √w—indicates weak binding to immobilized ligand on BIAcore chip; M—indicates monomer; D—indicates dimer; M/D—indicates monomer in equilibrium with dimer; D/T indicates the presence of dAb dimers and trimers in a sample; *—indicates that M/D not in equilibrium, tends more towards monomer).

Conclusion: Mutations at P44 alter the in solution state of DOM7h-8. A number of mutations monomerise the dimeric DOM7h-8.

### 2B) DOM 7h-14

For DOM7h-14, 3 individual libraries were made with mutations at former V_{H}/V_{L} interface residues, Q38, A43 and P44. Mutations were introduced by site-directed-mutagenesis using DOM7h-14 in the E. coli expression vector pDOM5 as a template and the NNK codon as described above. The primers were as follows:

Q38
(primers:
(SEQ ID NO: 37)
5′-GGGTCTCAGTTATCTTGGTACCAGNNKAAACCAGGGAAAGCCCC-
3′;
(SEQ ID NO: 38))
5′-GGGGCTTTCCCTGGTTTMNNCTGGTACCAAGATAACTGAGACCC-3′
A43
(primers:
(SEQ ID NO: 39)
5′-CAGCAGAAACCAGGGAAANNKCCTAAGCTCCTGATCATGTGG-3′;
(SEQ ID NO: 40))
5′-CCACATGATCAGGAGCTTAGGMNNTTTCCCTGGTTTCTGCTG-3′;
or
P44
(primers:
(SEQ ID NO: 41)
5′-CAGCAGAAACCAGGGAAAGCCNNKAAGCTCCTGATCATGTGGCGTTC
C-3′;
(SEQ ID NO: 42))
5′-GGAACGCCACATGATCAGGAGCTTMNNGGCTTTCCCTGGTTTCTGCT
G-3′

Libraries were transformed into E. coli HB2151 cells for screening.

Isolation of all amino acid variants at positions Q38, A43 or P44 in DOM7h-14: A colony screen PCR with primers DOM8 (SEQ ID NO: 16 as defined hereinbefore) and DOM9 (CGCCAGGGTTTTCCCAGTCACGAC (SEQ ID NO: 75)) was performed on 96 randomly picked clones from the DOM7h-14 libraries mutagenised at positions Q38, A43 or P44. PCR products were sequenced with DOM8 (SEQ ID NO: 16 as defined hereinbefore) and Protein L binding analysed to confirm that all dAbs are expressed and they retain Protein L binding.

Those clones missing from the initial screening effort were made by site-directed-mutagenesis with the following primers (Table 7):

TABLE 7
Primer pairs for site directed mutagenesis
to generate DOM7h-14 mutants not identified
in the NNK libraries at positions Q38, A43
or P44

| | |
|---|---|
| A43D | CAGCAGAAACCAGGGAAAGATCCTAAGCTCCTGATCATGTGG; |
| | (SEQ ID NO: 43) |
| | CCACATGATCAGGAGCTTAGGATCTTTCCCTGGTTTCTGCTG |
| | (SEQ ID NO: 44) |
| A43E | CAGCAGAAACCAGGGAAAGAACCTAAGCTCCTGATCATGTGG; |
| | (SEQ ID NO: 45) |
| | CCACATGATCAGGAGCTTAGGTTCTTTCCCTGGTTTCTGCTG |
| | (SEQ ID NO: 46) |
| P44Q | CAGCAGAAACCAGGGAAAGCCCAGAAGCTCCTGATCATGTGGCGTT |
| | CC |
| | (SEQ ID NO: 47) |
| | GGAACGCCACATGATCAGGAGCTTCTGGGCTTTCCCTGGTTTCTGC |
| | TG |
| | (SEQ ID NO: 48) |
| P44I | CAGCAGAAACCAGGGAAAGCCATTAAGCTCCTGATCATGTGGCGTT |
| | CC |
| | (SEQ ID NO: 49) |
| | GGAACGCCACATGATCAGGAGCTTAATGGCTTTCCCTGGTTTCTGC |
| | TG |
| | (SEQ ID NO: 50) |
| P44M | CAGCAGAAACCAGGGAAAGCCATGAAGCTCCTGATCATGTGGCGTT |
| | CC |
| | (SEQ ID NO: 51) |
| | GGAACGCCACATGATCAGGAGCTTCATGGCTTTCCCTGGTTTCTGC |
| | TG |
| | (SEQ ID NO: 52) |
| P44F | CAGCAGAAACCAGGGAAAGCCTTTAAGCTCCTGATCATGTGGCGTT |
| | CC |
| | (SEQ ID NO: 53) |
| | GGAACGCCACATGATCAGGAGCTTAAAGGCTTTCCCTGGTTTCTGC |
| | TG |
| | (SEQ ID NO: 54) |
| P44 | CAGCAGAAACCAGGGAAAGCCTGGAAGCTCCTGATCATGTGGCGTT |
| W | CC |
| | (SEQ ID NO: 55) |
| | GGAACGCCACATGATCAGGAGCTTCCAGGCTTTCCCTGGTTTCTGC |
| | TG |
| | (SEQ ID NO: 56) |

Screening of DOM7h-14 mutants: In order to characterize the potential of mutations at Q38, A43 and P44 to impact on the structure of a Vκ dAb and hence antigen-binding activity, all amino acid variants of a monomeric Vk dAb DOM7h-14 at positions Q38, A43 and P44 were BIAcore screened for Protein L and HSA binding activity. Binding to Protein L present on a separate flow cell on the same chip confirmed that dAb expression had occurred or was not compromised.

Screening of expressed supernatants for Protein L and HSA binding: Mutant clones were picked into a 96 well plate format and expressed in 1 mL 2×TY 0.1 mg/ml carbenicillin supplemented with OnEx solutions 1, 2 and 3 according to the manufacturer's instructions (Novagen). Cultures were grown at 30° C. for 3 days at 950 rpm high humidity in an InforsHT shaker. Cells were pelleted by centrifugation (4.5 k in a bench top Sorvall centrifuge for 30 mins) and 75 μL of the supernatant added to an equal volume of HBS-EP buffer (GE Healthcare). Expressed supernatants were screened by BIAcore for Protein L binding using Protein L (Sigma) coupled to a CM5 BIAcore chip (789 RU) and HSA coupled on a separate flow cell on the same CM5 chip (6036 RU) (see Table 8).

TABLE 8
BIAcore analysis of DOM7h-14 expressed supernatants for Protein L and antigen (HSA) binding.

| | Supernatant | Supernatant | | Supernatant | Supernatant | | Supernatant | Supernatant |
|---|---|---|---|---|---|---|---|---|
| Mutant | antigen binding | Protein L binding | Mutant | antigen binding | Protein L binding | Mutant | antigen binding | Protein L binding |
| 7h14 wt | ✓ | ✓ | 7h14 wt | ✓ | ✓ | 7h14 wt | ✓ | ✓ |
| Q38A | ✓ | ✓ | A43R | ✓ | ✓ | P44A | ✓ | ✓ |
| Q38R | ✓ | ✓ | A43N | ✓ | ✓ | P44R | ✓ | ✓ |
| Q38N | ✓ | ✓ | A43D | nd | nd | P44N | ✓ | ✓ |
| Q38D | ✓ | ✓ | A43C | ✓ | ✓ | P44D | ✓ | ✓ |
| Q38C | ✓ | ✓ | A43Q | nd | nd | P44C | ✓ | ✓ |
| Q38E | ✓ | ✓ | A43E | ✓ | ✓ | P44Q | ✓ | ✓ |
| Q38G | ✓ | ✓ | A43G | ✓ | ✓ | P44E | ✓ | ✓ |
| Q38H | ✓ | ✓ | A43H | ✓ | ✓ | P44G | ✓ | ✓ |
| Q38I | ✓ | ✓ | A43I | ✓ | ✓ | P44H | ✓ | ✓ |
| Q38L | ✓ | ✓ | A43L | ✓ | ✓ | P44I | ✓ | ✓ |
| Q38K | ✓ | ✓ | A43K | ✓ | ✓ | P44L | ✓ | ✓ |
| Q38M | ✓ | ✓ | A43M | ✓ | ✓ | P44K | ✓ | ✓ |
| Q38F | ✓ | ✓ | A43F | ✓ | ✓ | P44M | ✓ | ✓ |
| Q38P | ✓ | ✓ | A43P | ✓ | ✓ | P44F | nd | nd |
| Q38S | ✓ | ✓ | A43S | ✓ | ✓ | P44S | ✓ | ✓ |
| Q38T | ✓ | ✓ | A43T | ✓ | ✓ | P44T | ✓ | ✓ |
| Q38W | ✓ | ✓ | A43W | nd | nd | P44W | ✓ | ✓ |
| Q38Y | ✓ | ✓ | A43Y | ✓ | ✓ | P44Y | nd | nd |
| Q38V | ✓ | ✓ | A43V | ✓ | ✓ | P44V | ✓ | ✓ |

(✓ - indicates binding; nd—indicates not determined).

Conclusion: All mutants tested bind Protein L and retain HSA binding indicating that the mutations do not affect dAb structure and therefore antigen binding.

### Example 3

### Screening of PEP1-5-19 P44 Mutants

To determine the effect of making mutations in another clone, mutations at P44 in PEP1-5-19 were made by site-directed-mutagenesis using PEP1-5-19 in the E. coli expression vector pDOM5 as a template with primers

(SEQ ID NO: 57)
5′-GCAGAAACCAGGGAAAGCCNNKAAGCTCCTGATCTATAGTGC-3′,
(SEQ ID NO: 58)
5′-GCACTATAGATCAGGAGCTTMNNGGCTTTCCCTGGTTTCTGC-3′.

The parent PEP1-5-19 and 94 randomly picked colonies from the PEP1-5-19 P44 library were expressed in 1 mL 2×TY 0.1 mg/ml carbenicillin supplemented with OnEx solutions 1, 2 and 3 according to the manufacturer's instructions (Novagen) in a 96 well plate format. Cultures were grown at 30° C. for 3 days at 950 rpm high humidity in an InforsHT shaker. Cells were pelleted by centrifugation (4.5 k rpm in bench top Sorvall centrifuge for 30 mins) and 75 μL of the supernatant added to an equal volume of HBS-EP buffer (GE Healthcare). Expressed supernatants were screened by BIAcore for Protein L binding (311 RU) using Protein L (Sigma) coupled to a CM5 BIAcore chip. All clones were sequenced with primer DOM8 (SEQ ID NO: 16 as defined hereinbefore). Those clones that dissociated from Protein L significantly faster than the parent molecule PEP1-5-19 (a dimer) were assigned to be either stable monomers or monomers in equilibrium with dimers (see Table 9).

TABLE 9
Supernatant screen of PEP1-5-19 mutants at P44 for Protein L binding
(D-indicates dimer; M-indicates monomer; M/D-indicates
monomer/dimer; nd—not determined
because mutant not identified in the 94 clones sequenced).

| | Supernatant |
|---|---|
| Clone | Protein L Binding |
| PEP1-5-19 | D |
| P44A | M |
| P44R | M |
| P44N | M |
| P44D | Nd |
| P44C | Nd |
| P44Q | M |
| P44E | M |
| P44G | M |
| P44H | M |
| P44I | M/D |
| P44L | M/D |
| P44K | M |
| P44M | M |
| P44F | M/D |
| P44S | M |
| P44T | M |
| P44W | M |
| P44Y | M |
| P44V | M/D |

Conclusion: As was seen with mutants of DOM7h-8 at position P44, mutations altered the in solution state of the formerly dimeric PEP1-5-19.

### Example 4

### Construction of Pools of Naïve V_{κ} dAbs Mutated at Position 43

In order to develop further understanding of the potential for mutations at the former V_{H}/V_{L} interface to enhance the monomeric content of a dAb library in the context of a naïve library, the 4 G Vₖ dAb library (described in WO2005093074) was taken and mutations at position 43 were introduced by site directed mutagenesis. This approach permits analysis of mutations in a universal or broader context suggestive that a particular mutation will be effective across a wide range of CDR combinations and compositions.

Primers were designed by Stratagene Quikchange primer design software, to change Fw 2 position 43 to either A43A, -D, -K, -R, -E, -I or -L and synthesised by Sigma (synthesised to OD 1 μmol scale and purified by PAGE).

### Primer Sequences:

A43A_fwd:
(SEQ ID NO: 59)
gcagaaaccagggaaagcccctaagctcctgatc
A43A_rev:
(SEQ ID NO: 60)
gatcaggagcttaggggctttccctggtttctgc
A43D_fwd:
(SEQ ID NO: 61)
gcagaaaccagggaaagaccctaagctcctgatc
A43D_rev:
(SEQ ID NO: 62)
gatcaggagcttagggtctttccctggtttctgc
A43K_fwd:
(SEQ ID NO: 63)
aaattggtaccagcagaaaccagggaaaaagcctaagctcctgatc
A43K_rev:
(SEQ ID NO: 64)
gatcaggagcttaggctttttccctggtttctgctggtaccaattt
A43R_fwd:
(SEQ ID NO: 65)
gtaccagcagaaaccagggaaacggcctaagctcctg
A43R_rev:
(SEQ ID NO: 66)
caggagcttaggccgtttccctggtttctgctggtac
A43E_fwd:
(SEQ ID NO: 67)
cagcagaaaccagggaaagagcctaagctcctgatctatg
A43E_rev:
(SEQ ID NO: 68)
catagatcaggagcttaggctctttccctggtttctgctg
A43I_fwd:
(SEQ ID NO: 69)
ggtaccagcagaaaccagggaaaatccctaagctcct
A43I_rev:
(SEQ ID NO: 70)
aggagcttagggattttccctggtttctgctggtacc
A43L_fwd:
(SEQ ID NO: 71)
tggtaccagcagaaaccagggaaactgcctaagctcctga
A43L_rev:
(SEQ ID NO: 72)
tcaggagcttaggcagtttccctggtttctgctggtacca

Inoculated 50 ml 2×TY medium+carbencillin 100 μg/ml with 50 μl naïve 4 G V_{κ} library in pDOM10 glycerol stock, incubated 250 rpm, 37° C. overnight. Plasmid DNA was isolated using Qiagen QIAfilter midi-prep, in accordance with the manufacturer's instructions. pDOM10 is a plasmid vector, designed for soluble expression of dAbs. It is based on pUC119 vector, with expression under the control of the LacZ promoter. Expression of dAbs into the supernatant was ensured by fusion of the dAb gene to the universal GAS leader signal peptide (see WO2005093074) at the N-terminal end. In addition, a FLAG-tag was appended at the C-terminal end of the dAbs.)

Site directed mutagenesis reactions were done with the Stratagene Quikchange II kit, following the manufacturer's protocol except where indicated below. Reactions were carried out as follows: (per 50 μl reaction) 5 μl 10×reaction buffer, 1.55 μl (120 ng) pDOM10 naïve 4 G V_{κ} midiprep, 1.25 μl fwd primer (125 ng), 1.25 μl rev primer (125 ng), 1 μl dNTP mix, 38.95 μl sterile water, 1 μl Pfu ultra. Mutagenesis was performed with the following PCR program—1. 95° C. 30 s, 2. 95° C. 30 s, 3. 55° C. 1 min, 4. 68° C. 4 min, 5. To step 2×17 cycles, 6. 4° C. hold. 1 μl Dpn I was added to each reaction and incubated at 37° C. for 1 h.

5 μl of each Dpn I-digested reaction was transformed by mixing with 50 μl aliquots of electrocompetent HB2151 E. coli cells, incubating on ice for 30 min in 0.2 cm electroporation cuvettes (Biorad) and electroporating with standard E. coli K12 settings (2.5 kV/cm, 25 μF, 200Ω). 950 μl warmed SOC medium (Invitrogen, 15544-034) was added immediately following electroporation, transferred to a 14 ml Falcon tube and incubated at 37° C., 200 rpm for 1 h. The entire recovery cultures were plated (330 μl×3) to LB+carbencillin 100 μg/ml and incubated at 37° C. overnight. Clones were picked into 96 well plates (Corning) containing 125 μl 2×TY+2% glucose+100 μg/ml carbencillin, using a QPix2XT (Genetix) and incubated at 37° C., 250 rpm, overnight in a humidified incubator (New Brunswick).

Expression cultures were set up for two plates from each library pool: 1 ml TB+separate OnEx (Invitrogen) components (per 1 L medium: 20 ml solution 1, 50 ml solution 2, 1 ml solution 3)+carbenicillin 100 μg/ml+2 drops antifoam (A204, Sigma) added to 2 ml deep well block. Cultures were incubated 30° C., 750 rpm, 85% humidity for 3 days. Crude supernatant was then harvested and clarified by centrifugation at 4500 rpm, 4° C., 30 min and stored −80° C.

### Example 5

### Ranking the Monomerising Potential, Expression and Stability Effects of A43D, -K, -R, -E, -I and -L in a Naïve Library Background

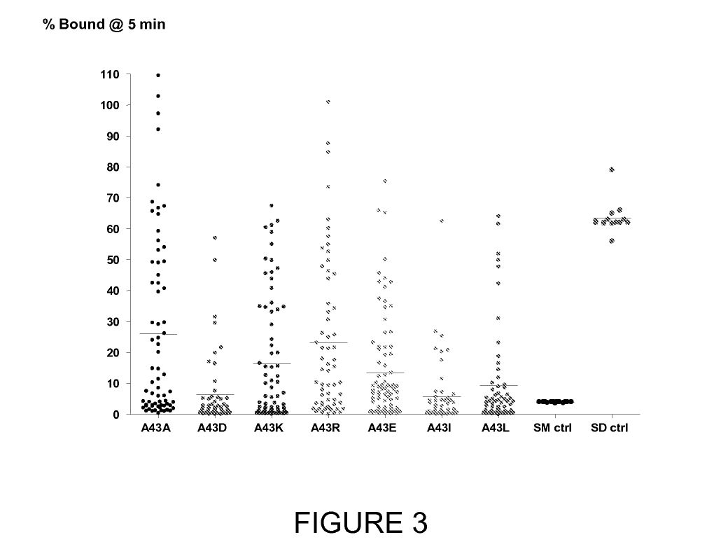

Undiluted, crude supernatant samples generated from the A43 mutant libraries described above were analysed by Protein L binding using a BIAcore 3000 instrument (BIAcore, Sweden), as described in the method section above. Two separate BIAcore CM5 chips were used to collect the data; both were derivatised with low amounts (˜500-700 RU) of Protein L in flowcells 2 and 3 (Fc2 and Fc3) and having a blank, activated-deactivated surface in flowcell 1 (Fc1). The results are shown in FIG. 3.

Data analysis was done using the report point tables from Fc2-1 or Fc 3-1, which were exported into Microsoft Excel. Two report points were included in the method, as described above and % B₅ values were generated. These % B₅ values were used to rank clones. The % B₅ values for control dAbs DOM7h-8 (dimer control, 64%±5) and DOM4-130-54 (monomer control, 4%±0.1) were used to categorise clones as monomer—(SM), dimer—(SD) or rapid-equilibrium-like (RE).

The amino acid and nucleic acid sequence for DOM4-130-54 is as follows:

### DOM4-130-54

Nucleotide sequence:
(SEQ ID NO: 73)
ATGTTATTTAAATCATTATCAAAATTAGCAACCGCAGCAGCATTTTTTGC
AGGCGTGGCAACAGCGTCGACGGACATCCAGATGACCCAGTCTCCATCCT
CCCTGTCTGCATCTGTAGGAGACCGTGTCACCATCACTTGCCGGGCAAGT
CAGGATATTTACCTGAATTTAGACTGGTATCAGCAGAAACCAGGGAAAGC
CCCTAAGCTCCTGATCAATTTTGGTTCCGAGTTGCAAAGTGGTGTCCCAT
CACGTTTCAGTGGCAGTGGATATGGGACAGATTTCACTCTCACCATCAGC
AGTCTGCAACCTGAAGATTTCGCTACGTACTACTGTCAACCGTCTTTTTA
CTTCCCTTATACGTTCGGCCAAGGGACCAAGGTGGAAATCAAACGGGCGG
CCGCAGAACAAAAACTCATCTCAGAAGAGGATCTGAATTAATAA
Amino acid sequence:
(SEQ ID NO: 74)
MLFKSLSKLATAAAFFAGVATASTDIQMTQSPSSLSASVGDRVTITCRAS
QDIYLNLDWYQQKPGKAPKLLINFGSELQSGVPSRFSGSGYGTDFTLTIS
SLQPEDFATYYCQPSFYFPYTFGQGTKVEIKRA

Clones were excluded from the spreadsheet analysis if: Response 1=<50 RU and Response 2=negative value or Response 1=negative value or sequencing showed that the identity of residue 43 was A (in the case of libraries where this should have been changed by SDM) or sequencing showed a putative unpaired Cysteine residue present in the dAb.

TABLE 10
Summary of statistics for FIG. 2.0, calculated by GraphPad Prism software.

| | A43A | A43D | A43K | A43R | A43E | A43I | A43L | SM ctrl | SD ctrl |
|---|---|---|---|---|---|---|---|---|---|
| Total number of values | 6 | 5 | 7 | 6 | 8 | 5 | 6 | 1 | 1 |
| Number of excluded values | 0 | 0 | 0 | 0 | 0 | 0 | 0 | 0 | 0 |
| Number of binned values | 6 | 5 | 7 | 6 | 8 | 5 | 6 | 1 | 1 |
| Minimum | 0.3 | 0.1 | 0.1 | 0.1 | 0.1 | 0.1 | 0.0 | 3.6 | 56.0 |
| 25% Percentile | 2.8 | 0.7 | 1.0 | 3.0 | 1.0 | 0.5 | 0.7 | 3.9 | 61.8 |
| Median | 10.8 | 2.0 | 5.6 | 14.2 | 7.1 | 1.4 | 3.7 | 4.0 | 62.0 |
| 75% Percentile | 47.0 | 5.2 | 33.4 | 39.7 | 20.2 | 5.2 | 8.6 | 4.0 | 64.0 |
| Maximum | 109. | 57.0 | 67.4 | 100. | 75.3 | 62.4 | 64.0 | 4.0 | 79.0 |
| Mean | 25.9 | 6.4 | 16.3 | 23.1 | 13.3 | 5.7 | 9.3 | 3.9 | 63.4 |
| Std. Deviation | 29.3 | 11.6 | 20.0 | 25.0 | 16.7 | 10.6 | 15.5 | 0.1 | 4.9 |
| Std. Error | 3.5 | 1.5 | 2.3 | 3.1 | 1.8 | 1.4 | 1.9 | 0.0 | 1.3 |
| Lower 95% CI of mean | 18.8 | 3.2 | 11.6 | 16.7 | 9.7 | 2.8 | 5.3 | 3.8 | 60.5 |
| Upper 95% CI of mean | 33.0 | 9.5 | 20.9 | 29.5 | 16.9 | 8.7 | 13.2 | 4.0 | 66.3 |

Conclusions: The Protein L BIAcore screen appeared to reveal differences in Protein L binding between the A43 libraries. Using both the summary graph and table (FIG. 3, Table 10) and visual inspection of the sensorgrams, general trends in the data across each library can be determined. Enrichment in monomer-like binding profiles was seen most clearly with the A43D, A43I and A43L libraries—this indicated that substituting or mutating residue A43 to either of these residues results in a library containing an enriched monomer population. A smaller reduction in mean % B₅ values was seen with the A43K and A43E libraries, whereas the A43R library generated a value equivalent to WT (A43A).

The SD (DOM7h8) and SM (DOM4-130-54) controls showed very reproducible % B₅ values across the 14 plates analysed, suggesting that the BIAcore chips used were retaining their binding capacity over many regeneration cycles.

### Example 6

### DOM7h-8 Mutants at Y36, L46 or Y87

For DOM7h-8, a further 3 further libraries were made with mutations at former VH/VL interface residues: Y36, L46 and Y87. Mutations were introduced by site-directed mutagenesis as described in Example 2A with the following primers:

Y36 (primers:
(SEQ ID NO: 76)
5′-GCAGCTATTTAAATTGGNNKCAGCAGAAACCAGGGAAAGCCCCTAA
G-3′;
(SEQ ID NO: 77))
5′-CTTAGGGGCTTTCCCTGGTTTCTGCTGMNNCCAATTTAAATAGCTG
C-3′
L46 (primers:
(SEQ ID NO: 78)
5′-CCAGGGAAAGCCCCTAAGNNKCTGATCTATCGGAATTCCCCTTT
G-3′;
(SEQ ID NO: 79))
5′-CAAAGGGGAATTCCGATAGATCAGMNNCTTAGGGGCTTTCCCTG
G-3′
Y87 (primers:
(SEQ ID NO: 80)
5′-CCTGAAGATTTTGCTACGTACNNKTGTCAACAGACGTATAG-3′;
(SEQ ID NO: 81))
5′-CTATACGTCTGTTGACAMNNGTACGTAGCAAAATCTTCAGG-3′

The NNK codon used to introduce diversity encodes all 20 amino acids and the TAG stop codon. Colonies were picked at random from each library and a colony PCR screen performed with primers DOM8 and DOM9 (as defined hereinbefore). Briefly a single colony was picked with a toothpick and dipped into a PCR mix comprising 23 μl of Platinum Blue PCR Supermix, 1 μl DOM8 (10 μM) and 1 μl DOM9 (10 μM). Reactions were thermocycled in an Eppendorf Mastercycler Gradient as follows: 95° C. 5 min; 30×(95° C. 30 sec, 55° C. 30 sec, 72° C. 1 min 30 sec). Colonies that were screened were either replica plated onto 2×TY Carb (0.1 mg/ml) agar plates and grown overnight at 37° C. or were inoculated into 100 μl 2×TY Carb (0.1 mg/ml) and grown overnight at 37° C., 250 rpm in an Infors HT shaker.

In order to obtain the full complement of amino acid variation at positions Y36, L46 and Y87 clones not identified in the random screening of the library were made by site-directed-mutagenesis using DOM7h-8 in the E. coli expression vector pDOM5 as a template with primers listed in Table 11.

TABLE 11
Primers for making Y36, L46 and Y87 mutants
not found during random screening

| | |
|---|---|
| Y87C | 5′-CCTGAAGATTTTGCTACGTACTGCTGTCAACAGACGTATAG- |
| | 3′ |
| | (SEQ ID NO: 82) |
| | 5′-CTATACGTCTGTTGACAGCAGTACGTAGCAAAATCTTCAGG- |
| | 3′ |
| | (SEQ ID NO: 83) |
| Y87E | 5′-CCTGAAGATTTTGCTACGTACGAATGTCAACAGACGTATAG- |
| | 3′ |
| | (SEQ ID NO: 84) |
| | 5′-CTATACGTCTGTTGACATTCGTACGTAGCAAAATCTTCAGG- |
| | 3′ |
| | (SEQ ID NO: 85) |
| Y87N | 5′-CCTGAAGATTTTGCTACGTACAACTGTCAACAGACGTATAG- |
| | 3′ |
| | (SEQ ID NO: 86) |
| | 5′-CTATACGTCTGTTGACAGTTGTACGTAGCAAAATCTTCAGG- |
| | 3′ |
| | (SEQ ID NO: 87) |
| Y87D | 5′-CCTGAAGATTTTGCTACGTACGATTGTCAACAGACGTATAG- |
| | 3′ |
| | (SEQ ID NO: 88) |
| | 5′-CTATACGTCTGTTGACAATCGTACGTAGCAAAATCTTCAGG- |
| | 3′ |
| | (SEQ ID NO: 89) |
| Y87K | 5′-CCTGAAGATTTTGCTACGTACAAATGTCAACAGACGTATAG- |
| | 3′ |
| | (SEQ ID NO: 90) |
| | 5′-CTATACGTCTGTTGACATTTGTACGTAGCAAAATCTTCAGG- |
| | 3′ |
| | (SEQ ID NO: 91) |
| Y87P | 5′-CCTGAAGATTTTGCTACGTACCCATGTCAACAGACGTATAG- |
| | 3′ |
| | (SEQ ID NO: 92) |
| | 5′-CTATACGTCTGTTGACACGGGTACGTAGCAAAATCTTCAGG- |
| | 3′ |
| | (SEQ ID NO: 93) |
| L46C | 5′- |
| | CCAGGGAAAGCCCCTAAGTGCCTGATCTATCGGAATTCCCCTTTG- |
| | 3′ |
| | (SEQ ID NO: 94) |
| | 5′- |
| | CAAAGGGGAATTCCGATAGATCAGGCACTTAGGGGCTTTCCCTG |
| | G-3′ |
| | (SEQ ID NO: 95) |
| Y36D | 5′- |
| | GCAGCTATTTAAATTGGGATCAGCAGAAACCAGGGAAAGCCCCT |
| | AAG-3′ |
| | (SEQ ID NO: 96) |
| | 5′- |
| | CTTAGGGGCTTTCCCTGGTTTCTGCTGATCCCAATTTAAATAGCTG |
| | C-3′ |
| | (SEQ ID NO: 97) |
| Y36N | 5′- |
| | GCAGCTATTTAAATTGGAACCAGCAGAAACCAGGGAAAGCCCCT |
| | AAG-3′ |
| | (SEQ ID NO: 98) |
| | 5′- |
| | CTTAGGGGCTTTCCCTGGTTTCTGCTGGTTCCAATTTAAATAGCTG |
| | C-3′ |
| | (SEQ ID NO: 99) |
| Y36M | 5′- |
| | GCAGCTATTTAAATTGGATGCAGCAGAAACCAGGGAAAGCCCCT |
| | AAG-3′ |
| | (SEQ ID NO: 100) |
| | 5′- |
| | CTTAGGGGCTTTCCCTGGTTTCTGCTGCATCCAATTTAAATAGCTG |
| | C-3′ |
| | (SEQ ID NO: 101) |

DOM7h-8 mutants at positions Y36, L46 or Y87 were screened as purified proteins by BIAcore in order to characterize dAb binding activity to HSA and superantigen Protein L.

Protein from all clones expressing mutants of DOM7h-8 at positions Q38, A43 or P44 was expressed in 50 ml cultures in 2×TY Carbenicillin 100 μg/ml, antifoam, supplemented with OnEx solutions 1, 2 and 3 according to the manufacturer's instructions (Novagen). Cultures were grown at 30° C. for 3 days at 250 rpm in an InforsHT shaker at 250 rpm. Cells were pelleted by centrifugation (4.5 k in a bench top Sorvall centrifuge for 30 mins) the expressed dAb was purified from the supernatant by affinity chromatography to Protein L using a PCC48 (The Automation Partnership).

Purified proteins at, wherever possible, 1 μM were assayed by BIAcore for binding to Protein L (311 RU) and binding to HSA (559 RU) coupled to separate flow cells on a CM5 chip. Those clones that dissociated from Protein L significantly faster than the parent molecule DOM7h-8 (a dimer) were assigned to be either stable monomers or monomers in equilibrium with dimers. Purified proteins were also analysed for HSA binding to assess the effect mutations have on the conformation of CDR regions of the dAb that make contact with antigen (see Table 12).

TABLE 12
BIAcore analysis of DOM7h-8 purified protein for Protein L
and antigen (HSA) binding

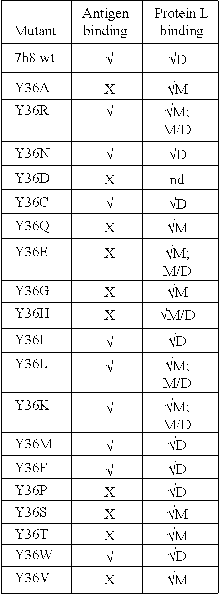

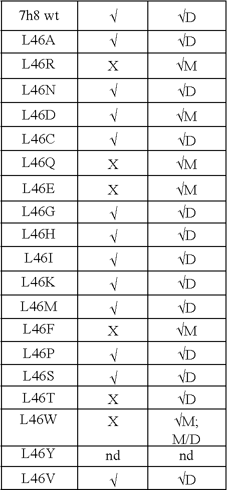

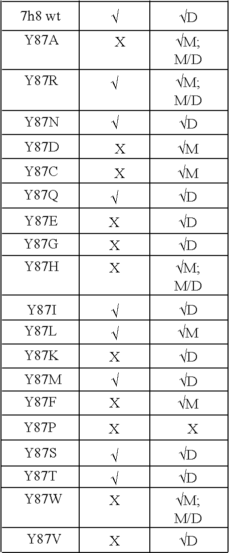

(√— indicates binding; X indicates no binding;
M indicates monomer;
D indicates dimer;
M/D indicates monomer in equilibrium with dimer;
nd indicates not determined).
Mutants highlighted in general monomerise and disrupt HSA binding, but mutants L46D and Y87L retain antigen binding and form stable monomers.

Conclusion: Some mutants of DOM7h-8 parent dAb molecule no longer bind HSA but nevertheless maintain the dimeric state of the parent, as based on Protein L binding results. This suggests that these mutations apparently disrupt the HSA-binding paratope conformation without affecting the integrity of the protein L-binding site or the dimerisation state of the molecule. Several mutations at Y36, L46 or Y87 appear to monomerise DOM7h-8. Mutants L46D and Y87L were found to cause monomerisation of DOM7h-8 and retained HSA binding.

### Example 7

The A43I and A43D mutations were introduced into DOM7h-11-15 by site-directed mutagenesis using DOM7h-11-15 in the E. coli expression vector pET30a as a template with the primers listed below:

A43I (primers:
(SEQ ID NO: 102)
5′-CAGCAGAAACCAGGGAAAATTCCTAAGCTCCTGATCCTT-3′
(SEQ ID NO: 103))
5′-AAGGATCAGGAGCTTAGGAATTTTCCCTGGTTTCTGCTG-3′
A43D (primers:
(SEQ ID NO: 104)
5′-CAGCAGAAACCAGGGAAAGATCCTAAGCTCCTGATCCTT-3′
(SEQ ID NO: 105))
5′-AAGGATCAGGAGCTTAGGATCTTTCCCTGGTTTCTGCTG-3′

The amino acid and nucleic acid sequence for DOM7h-11-15 is as follows:

DOM7h-11-15 nucleotide sequence:
(SEQ ID NO: 106)
GACATCCAGATGACCCAGTCTCCATCCTCCCTGTCTGCATCTGTAGGAGA
CCGTGTCACCATCACTTGCCGGGCAAGTCGTCCGATTGGGACGATGTTAA
GTTGGTACCAGCAGAAACCAGGGAAAGCCCCTAAGCTCCTGATCCTTGCT
TTTTCCCGTTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGCGCGCAGGCTGGGACGCATCCTACGACGTTCGGCCAA
GGGACCAAGGTGGAAATCAAACGG
DOM7h-11-15 amino acid sequence:
(SEQ ID NO: 107)
DIQMTQSPSSLSASVGDRVTITCRASRPIGTMLSWYQQKPGKAPKLLILA
FSRLQSGVPSRFSGSGSGTDFTLTISSLQPEDFATYYCAQAGTHPTTFGQ
GTKVEIKR

The A43I and A43D mutations were introduced into DOM7h-14-10 by site-directed mutagenesis using DOM7h-14-10 in the E. coli expression vector pET30a as a template with the primers listed below:

A43I (primers:
(SEQ ID NO: 108)
5′-CAGCAGAAACCAGGGAAAATTCCTAAGCTCCTGATCATG-3′
(SEQ ID NO: 109))
5′-CATGATCAGGAGCTTAGGAATTTTCCCTGGTTTCTGCTG-3′
A43D (primers:
(SEQ ID NO: 110)
5′-CAGCAGAAACCAGGGAAAGATCCTAAGCTCCTGATCATG-3′
(SEQ ID NO: 111))
5′-CATGATCAGGAGCTTAGGATCTTTCCCTGGTTTCTGCTG-3′

The amino acid and nucleic acid sequence for DOM7h-14-10 is as follows:

DOM7h-14-10 nucleotide sequence:
(SEQ ID NO: 112)
GACATCCAGATGACCCAGTCTCCATCCTCCCTGTCTGCATCTGTAGGAGA
CCGTGTCACCATCACTTGCCGGGCAAGTCAGTGGATTGGGTCTCAGTTAT
CTTGGTACCAGCAGAAACCAGGGAAAGCCCCTAAGCTCCTGATCATGTGG
CGTTCCTCGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTGCTCAGGGTTTGAGGCATCCTAAGACGTTCGGCCAA
GGGACCAAGGTGGAAATCAAACGG
DOM7h-14-10 amino acid sequence:
(SEQ ID NO: 113)
DIQMTQSPSSLSASVGDRVTITCRASQWIGSQLSWYQQKPGKAPKLLIMW
RSSLQSGVPSRFSGSGSGTDFTLTISSLQPEDFATYYCAQGLRHPKTFGQ
GTKVEIKR

Protein of DOM7h-11-15 parent and A43D or A43I mutants and the DOM7h-14-10 parent and A43D and A43I mutants was expressed and purified from E. coli cells using OnEx autoinduction system (Invitrogen, UK) in 2×TY medium. Binding of purified parent or mutant proteins to HSA was analysed on a Biacore 2000 with a low density CM5 chip to which was coupled 559 RU HSA (see Example methods). Proteins were analysed at 1 μM, 0.5 μM, 0.25 μM, 125 nM, 62 nM, 32 nM, 16 nM and 8 nM concentrations.

The K_{D} of DOM7h-11-15 is 3.8 nM and the K_{D} of the DOM7h-11-15 A43I mutant is 6.4 nM. The mutant has a 1000-fold improvement in antigen affinity over that of the monomeric DOM7h-11-15 parent. The monomeric status of the A43D and A43I mutants was established independently by analytical ultracentrifugation.

The K_{D} of DOM7h-14-10 is 26 nM and the K_{D} of the of the A43I and A43D mutants is 11.7 nM and 13.1 nM, respectively. The mutants have a 2-fold improvement in antigen affinity over that of the monomeric DOM7h-14-10. The monomeric status of the A43D and A43I mutants was established independently by analytical centrifugation.

TABLE 13
Results of binding analysis with purified parent or mutant proteins to HSA.

| dAb | kₒₙ (M⁻¹s⁻¹) | k_{off} (S⁻¹) | K_{D}, nM |
|---|---|---|---|
| DOM7h-14-10 | 6.7e5 | 0.017 | 26 |
| DOM7h-14-10 | 9.9e5 | 0.012 | 11.7 |
| A43D | | | |
| DOM7h-14-10 A43I | 8.9e5 | 0.012 | 13.1 |
| DOM7h-11-15 | 1.2e4 | 4.5e-3 | 384 |
| DOM7h-11-15 | 664 | 4.7e-3 | 7000 |
| A43D | | | |
| DOM7h-11-15 A43I | 7.7e5 | 4.9e-3 | 6.4 |

Conclusion: Surprisingly, mutations at the former interface of antibody variable domains have been shown to beneficially influence the paratope, thereby improving the antigen-binding affinity of domain antibodies.

### REFERENCES

Bathelemy et al., 2007. Comprehensive analysis of the factors contributing to the stability and solubility of autonomous human VH domains. J Biol Chem 283 p 3639-3654.
Chatellier et al., 1996. Functional mapping of the conserved residues located at the VL and VH domain interface of a Fab. J Mol Biol 246 p 1-6.
Chothia et al., 1985. Domain association in immunoglobulin molecules the packing of variable domains. J Mol Biol 186 651-663.
Famm et al., 2008. Thermodynamically stable aggregation resistant antibody domains through directed evolution. J Mol Biol 376 p 926-931.
Jespers et al., 2004. Aggregation-resistant domain antibodies selected on phage by heat denaturation. Nature Biotech 22 p 1161-1165.
Matsuura and Plückthun 2003. Selection based on the folding properties of proteins with ribosome display. FEBS 539 p 24-28.
Matsuura and Plückthun 2004. Strategies for selection from protein libraries composed of de novo designed secondary structure modules. Origins of life and evolution of the biosphere 34 p 151-157.
Raffen et al., 1998. Reengineering immunoglobulin domain interactions by introduction of charged residues. Protein Engineering 11 p 303-309.
Sieber et al., 1998. Selecting proteins with improved stability by a phage-based method. Nature 16 p 955-960.
Stevens et al., 1980. Self-association of the human immunoglobulin _{κ}I light chains: role of the third hypervariable region. PNAS 77 pe 1144-1148.
U.S. Pat. No. 6,485,943. Method for altering antibody light chain interactions.
Vargas-Madrazo and Paz-Garcia 2003. An improved model of association for VH-VL immunoglobulin domains: asymmetries between VH and VL in the packing of some of the interface residues. J Mol Recog 16 p 113-120.

1-35. (canceled)

36. An isolated polypeptide comprising a variant immunoglobulin light chain single variable domain wherein said variant comprises the amino acid sequence of a framework region encoded by a human germline antibody gene segment and wherein at least one of the amino acids at positions 36, 38, 43, 44, 46 and 87 has been replaced, said positions assigned in accordance with the Kabat amino acid numbering system.

37. An isolated polypeptide as claimed in claim 36 wherein said variant immunoglobulin light chain single variable domain is a human V_{L} immunoglobulin light chain single variable domain.

38. An isolated polypeptide as claimed in claim 36 wherein the variant is substantially dimeric in solution.

39. An isolated polypeptide as claimed in claim 38 wherein the variant has at least one of the following amino acids, Y36, Q38, A43, P44, L46 or Y87.

40. An isolated polypeptide as claimed in claim 36 wherein the variant is substantially monomeric in solution.

41. An isolated polypeptide as claimed in claim 40 wherein the variant comprises an amino acid sequence in which the amino acid Y36 has been replaced by any of the amino acids A, Q, G, S, T or V.

42. An isolated polypeptide as claimed in any of claim 40 wherein the variant comprises an amino acid sequence in which the amino acid A43 has been replaced by D, I, L, F, T or W.

43. An isolated polypeptide as claimed in any of claim 40 wherein the variant comprises an amino acid sequence in which the amino acid P44 has been replaced by R, N, D, C, Q, E, H, I, L, K, M, F, T, Y or V.

44. An isolated polypeptide as claimed in any of claim 37 wherein the V_{L} is a Kappa lineage V_{L} (Vκ), preferably a Kappa I lineage V_{L} and, most preferably, DPK9.

45. A list or library of polypeptides comprising the polypeptides or immunoglobulins as claimed in any of claim 36 wherein at least 70% of the polypeptides are in monomeric form.

46. A library comprising a polypeptide or variant immunoglobulin light chain variable domain region as claimed in any of claim 36 wherein at least one of amino acid positions 36, 38, 43, 44, 46 or 87 has been mutated, said positions being assigned in accordance with the Kabat amino acid numbering system, and preferably wherein position 43 is selected from D, I, L, K or E.

47. A library for expressing polypeptides or variant immunoglobulin light chain variable domain regions as claimed in any of claim 36 comprising a list of nucleic acid sequences encoding said polypeptides or immunoglobulin light chain variable domains.

48. A library of nucleic acids encoding a polypeptide or an immunoglobulin light chain single variable domain as claimed in any of claim 36.

49. A list or library as claimed in claim 45 wherein said library further comprises diversity in the CDR regions.

50. A nucleic acid encoding a polypeptide or immunoglobulin light chain single variable domain as claimed in any of claims 36.

51. A library as claimed in claim 46 wherein said library further comprises diversity in the CDR regions.


SEQUENCE LISTING (113 sequences)

SEQ ID NO: 1 - protein, 108 aa, Artificial
  PEP1-5-19 (anti-TNFalpha dAb
DIQMTQSPSSLSASVGDRVTITCRASQSIDSYLHWYQQKPGKAPKLLIYSASELQSGVPSRFSGSGSGTDFTLTISSLQPEDFATYYCQQVVWRPFTFGQGTKVEIKR

SEQ ID NO: 2 - protein, 108 aa, Artificial
  DOM15-10 (anti-human VEGF dAb)
DIQMTQSPSSLSASVGDRVTITCRASQWIGPELSWYQQKPGKAPKLLIYHTSILQSGVPSRFSGSGSGTDFTLTISSLQPEDFATYYCQQYMFQPRTFGQGTKVEIRR

SEQ ID NO: 3 - protein, 108 aa, Artificial
  DOM13-25-3 (anti-CEA dAb)
DIQMTQSPSSLSASVGDRVTITCRASQSIGPWLSWYQQKPGKAPKLLFYQVSRLQSGVPSRFSGSGSGTDFTLTIISLQPEDFATYYCQQNLAPPYTFGQGTKVEIKR

SEQ ID NO: 4 - protein, 108 aa, Artificial
  DOM9-155-25 (anti-IL-4 anti Fcn dAb)
DIQMTQSPSSLSASVGDRVTITCRASRPISDWLHWYQQKPGKAPKLLIAWASTLDSGVPSRFSGSGSGTDFTLTISSLQPEDFATYYCLQEGWGPPTFGQGTKVEIKR

SEQ ID NO: 5 - protein, 108 aa, Artificial
  DOM7h-14 (anti-HSA dAb)
DIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIKR

SEQ ID NO: 6 - DNA, 324 bp, Artificial
  DOM7h-8 Nucleotide-sequence
gacatccagatgacccagtctccatcctccctgtctgcatctgtaggagaccgtgtcaccatcacttgccgggcaagtcagagcattagcagctatttaaattggtatcagcagaaaccagggaaagcccctaagctcctgatctatcggaattcccctttgcaaagtggggtcccatcacgtttcagtggcagtggatctgggacagatttcactctcaccatcagcagtctgcaacctgaagattttgctacgtactactgtcaacagacgtatagggtgcctcctacgttcggccaagggaccaaggtggaaatcaaacgg

SEQ ID NO: 7 - protein, 108 aa, Artificial
  DOM7h-8 Amino acid-sequence
DIQMTQSPSSLSASVGDRVTITCRASQSISSYLNWYQQKPGKAPKLLIYRNSPLQSGVPSRFSGSGSGTDFTLTISSLQPEDFATYYCQQTYRVPPTFGQGTKVEIKR

SEQ ID NO: 8 - DNA, 324 bp, Artificial
  DOM7h-14 Nucleotide-sequence
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgg

SEQ ID NO: 9 - protein, 108 aa, Artificial; identical to SEQ ID NO: 5
  DOM7h-14 Amino acid-sequence

SEQ ID NO: 10 - DNA, 43 bp, Artificial
  DOM7h-8 Q38 Primer
gcagctatttaaattggtatcagnnkaaaccagggaaagcccc

SEQ ID NO: 11 - DNA, 43 bp, Artificial
  DOM7h-8 Q38 Primer
ggggctttccctggtttmnnctgataccaatttaaatagctgc

SEQ ID NO: 12 - DNA, 42 bp, Artificial
  DOM7h-8 A43 Primer
cagcagaaaccagggaaannkcctaagctcctgatctatcgg

SEQ ID NO: 13 - DNA, 42 bp, Artificial
  DOM7h-8 A43 Primer
ccgatagatcaggagcttaggmnntttccctggtttctgctg

SEQ ID NO: 14 - DNA, 49 bp, Artificial
  DOM7h-8 P44 Primer
cagcagaaaccagggaaagccnnkaagctcctgatctatcggaattccc

SEQ ID NO: 15 - DNA, 49 bp, Artificial
  DOM7h-8 P44 Primer
gggaattccgatagatcaggagcttmnnggctttccctggtttctgctg

SEQ ID NO: 16 - DNA, 24 bp, Artificial
  DOM7h-8 DOM8 Primer
agcggataacaatttcacacagga

SEQ ID NO: 17 - DNA, 43 bp, Artificial
  Q38C DOM7h-8 Primer
gcagctatttaaattggtatcagtgcaaaccagggaaagcccc

SEQ ID NO: 18 - DNA, 43 bp, Artificial
  Q38C DOM7h-8 Primer
ggggctttccctggtttgcactgataccaatttaaatagctgc

SEQ ID NO: 19 - DNA, 43 bp, Artificial
  Q38K DOM7h-8 Primer
gcagctatttaaattggtatcagaaaaaaccagggaaagcccc

SEQ ID NO: 20 - DNA, 43 bp, Artificial
  Q38K DOM7h-8 Primer
ggggctttccctggttttttctgataccaatttaaatagctgc

SEQ ID NO: 21 - DNA, 47 bp, Artificial
  A43N DOM7h-8 Primer
ggtatcagcagaaaccagggaaaaaccctaagctcctgatctatcgg

SEQ ID NO: 22 - DNA, 47 bp, Artificial
  A43N DOM7h-8 Primer
ccgatagatcaggagcttagggtttttccctggtttctgctgatacc

SEQ ID NO: 23 - DNA, 40 bp, Artificial
  A43D DOM7h-8 Primer
cagcagaaaccagggaaagatcctaagctcctgatctatc

SEQ ID NO: 24 - DNA, 40 bp, Artificial
  A43D DOM7h-8 Primer
gatagatcaggagcttaggatctttccctggtttctgctg

SEQ ID NO: 25 - DNA, 47 bp, Artificial
  A43C DOM7h-8 Primer
ggtatcagcagaaaccagggaaatgccctaagctcctgatctatcgg

SEQ ID NO: 26 - DNA, 47 bp, Artificial
  A43C DOM7h-8 Primer
ccgatagatcaggagcttagggcatttccctggtttctgctgatacc

SEQ ID NO: 27 - DNA, 47 bp, Artificial
  A43I DOM7h-8 Primer
ggtatcagcagaaaccagggaaaattcctaagctcctgatctatcgg

SEQ ID NO: 28 - DNA, 47 bp, Artificial
  A43I DOM7h-8 Primer
ccgatagatcaggagcttaggaattttccctggtttctgctgatacc

SEQ ID NO: 29 - DNA, 49 bp, Artificial
  P44C DOM7h-8 Primer
cagcagaaaccagggaaagcctgcaagctcctgatctatcggaattccc

SEQ ID NO: 30 - DNA, 49 bp, Artificial
  P44C DOM7h-8 Primer
gggaattccgatagatcaggagcttgcaggctttccctggtttctgctg

SEQ ID NO: 31 - DNA, 49 bp, Artificial
  P44E DOM7h-8 Primer
cagcagaaaccagggaaagccgaaaagctcctgatctatcggaattccc

SEQ ID NO: 32 - DNA, 49 bp, Artificial; identical to SEQ ID NO: 31
  P44E DOM7h-8 Primer

SEQ ID NO: 33 - DNA, 49 bp, Artificial
  P44T DOM7h-8 Primer
cagcagaaaccagggaaagccaccaagctcctgatctatcggaattccc

SEQ ID NO: 34 - DNA, 49 bp, Artificial
  P44T DOM7h-8 Primer
gggaattccgatagatcaggagcttggtggctttccctggtttctgctg

SEQ ID NO: 35 - DNA, 49 bp, Artificial
  P44W DOM7h-8 Primer
cagcagaaaccagggaaagcctggaagctcctgatctatcggaattccc

SEQ ID NO: 36 - DNA, 49 bp, Artificial
  P44W DOM7h-8 Primer
gggaattccgatagatcaggagcttccaggctttccctggtttctgctg

SEQ ID NO: 37 - DNA, 44 bp, Artificial
  DOM 7h-14 Q38 Primer
gggtctcagttatcttggtaccagnnkaaaccagggaaagcccc

SEQ ID NO: 38 - DNA, 44 bp, Artificial
  DOM 7h-14 Q38 Primer
ggggctttccctggtttmnnctggtaccaagataactgagaccc

SEQ ID NO: 39 - DNA, 42 bp, Artificial
  DOM 7h-14 A43 Primer
cagcagaaaccagggaaannkcctaagctcctgatcatgtgg

SEQ ID NO: 40 - DNA, 42 bp, Artificial
  DOM 7h-14 A43 Primer
ccacatgatcaggagcttaggmnntttccctggtttctgctg

SEQ ID NO: 41 - DNA, 48 bp, Artificial
  DOM 7h-14 P44 Primer
cagcagaaaccagggaaagccnnkaagctcctgatcatgtggcgttcc

SEQ ID NO: 42 - DNA, 48 bp, Artificial
  DOM 7h-14 P44 Primer
ggaacgccacatgatcaggagcttmnnggctttccctggtttctgctg

SEQ ID NO: 43 - DNA, 42 bp, Artificial
  A43D DOM7h-14 Primer
cagcagaaaccagggaaagatcctaagctcctgatcatgtgg

SEQ ID NO: 44 - DNA, 42 bp, Artificial
  A43D DOM7h-14 Primer
ccacatgatcaggagcttaggatctttccctggtttctgctg

SEQ ID NO: 45 - DNA, 42 bp, Artificial
  A43E DOM7h-14 Primer
cagcagaaaccagggaaagaacctaagctcctgatcatgtgg

SEQ ID NO: 46 - DNA, 42 bp, Artificial
  A43E DOM7h-14 Primer
ccacatgatcaggagcttaggttctttccctggtttctgctg

SEQ ID NO: 47 - DNA, 48 bp, Artificial
  P44Q DOM7h-14 Primer
cagcagaaaccagggaaagcccagaagctcctgatcatgtggcgttcc

SEQ ID NO: 48 - DNA, 48 bp, Artificial
  P44Q DOM7h-14 Primer
ggaacgccacatgatcaggagcttctgggctttccctggtttctgctg

SEQ ID NO: 49 - DNA, 48 bp, Artificial
  P44I DOM7h-14 Primer
cagcagaaaccagggaaagccattaagctcctgatcatgtggcgttcc

SEQ ID NO: 50 - DNA, 48 bp, Artificial
  P44I DOM7h-14 Primer
ggaacgccacatgatcaggagcttaatggctttccctggtttctgctg

SEQ ID NO: 51 - DNA, 48 bp, Artificial
  P44M DOM7h-14 Primer
cagcagaaaccagggaaagccatgaagctcctgatcatgtggcgttcc

SEQ ID NO: 52 - DNA, 48 bp, Artificial
  P44M DOM7h-14 Primer
ggaacgccacatgatcaggagcttcatggctttccctggtttctgctg

SEQ ID NO: 53 - DNA, 48 bp, Artificial
  P44F DOM7h-14 Primer
cagcagaaaccagggaaagcctttaagctcctgatcatgtggcgttcc

SEQ ID NO: 54 - DNA, 48 bp, Artificial
  P44F DOM7h-14 Primer
ggaacgccacatgatcaggagcttaaaggctttccctggtttctgctg

SEQ ID NO: 55 - DNA, 48 bp, Artificial
  P44W DOM7h-14 Primer
cagcagaaaccagggaaagcctggaagctcctgatcatgtggcgttcc

SEQ ID NO: 56 - DNA, 48 bp, Artificial
  P44W DOM7h-14 Primer
ggaacgccacatgatcaggagcttccaggctttccctggtttctgctg

SEQ ID NO: 57 - DNA, 42 bp, Artificial
  pDOM5 Primer
gcagaaaccagggaaagccnnkaagctcctgatctatagtgc

SEQ ID NO: 58 - DNA, 42 bp, Artificial
  pDOM5 Primer
gcactatagatcaggagcttmnnggctttccctggtttctgc

SEQ ID NO: 59 - DNA, 34 bp, Artificial
  A43A_fwd Primer
gcagaaaccagggaaagcccctaagctcctgatc

SEQ ID NO: 60 - DNA, 34 bp, Artificial
  A43A_rev Primer
gatcaggagcttaggggctttccctggtttctgc

SEQ ID NO: 61 - DNA, 34 bp, Artificial
  A43D_fwd Primer
gcagaaaccagggaaagaccctaagctcctgatc

SEQ ID NO: 62 - DNA, 34 bp, Artificial
  A43D_rev Primer
gatcaggagcttagggtctttccctggtttctgc

SEQ ID NO: 63 - DNA, 46 bp, Artificial
  A43K_fwd Primer
aaattggtaccagcagaaaccagggaaaaagcctaagctcctgatc

SEQ ID NO: 64 - DNA, 46 bp, Artificial
  A43K_rev Primer
gatcaggagcttaggctttttccctggtttctgctggtaccaattt

SEQ ID NO: 65 - DNA, 37 bp, Artificial
  A43R_fwd Primer
gtaccagcagaaaccagggaaacggcctaagctcctg

SEQ ID NO: 66 - DNA, 37 bp, Artificial
  A43R_rev Primer
caggagcttaggccgtttccctggtttctgctggtac

SEQ ID NO: 67 - DNA, 40 bp, Artificial
  A43E_fwd Primer
cagcagaaaccagggaaagagcctaagctcctgatctatg

SEQ ID NO: 68 - DNA, 40 bp, Artificial
  A43E_rev Primer
catagatcaggagcttaggctctttccctggtttctgctg

SEQ ID NO: 69 - DNA, 37 bp, Artificial
  A43I_fwd Primer
ggtaccagcagaaaccagggaaaatccctaagctcct

SEQ ID NO: 70 - DNA, 37 bp, Artificial
  A43I_rev Primer
aggagcttagggattttccctggtttctgctggtacc

SEQ ID NO: 71 - DNA, 40 bp, Artificial
  A43L_fwd Primer
tggtaccagcagaaaccagggaaactgcctaagctcctga

SEQ ID NO: 72 - DNA, 40 bp, Artificial
  A43L_rev Primer
tcaggagcttaggcagtttccctggtttctgctggtacca

SEQ ID NO: 73 - DNA, 444 bp, Artificial
  DOM4-130-54 Nucleotide sequence
atgttatttaaatcattatcaaaattagcaaccgcagcagcattttttgcaggcgtggcaacagcgtcgacggacatccagatgacccagtctccatcctccctgtctgcatctgtaggagaccgtgtcaccatcacttgccgggcaagtcaggatatttacctgaatttagactggtatcagcagaaaccagggaaagcccctaagctcctgatcaattttggttccgagttgcaaagtggtgtcccatcacgtttcagtggcagtggatatgggacagatttcactctcaccatcagcagtctgcaacctgaagatttcgctacgtactactgtcaaccgtctttttacttcccttatacgttcggccaagggaccaaggtggaaatcaaacgggcggccgcagaacaaaaactcatctcagaagaggatctgaattaataa

SEQ ID NO: 74 - protein, 133 aa, Artificial
  DOM4-130-54 Amino Acid sequence
MLFKSLSKLATAAAFFAGVATASTDIQMTQSPSSLSASVGDRVTITCRASQDIYLNLDWYQQKPGKAPKLLINFGSELQSGVPSRFSGSGYGTDFTLTISSLQPEDFATYYCQPSFYFPYTFGQGTKVEIKRA

SEQ ID NO: 75 - DNA, 24 bp, Artificial
  DOM9 Primer
cgccagggttttcccagtcacgac

SEQ ID NO: 76 - DNA, 47 bp, Artificial
  Y36 DOM7h-8 Primer
gcagctatttaaattggnnkcagcagaaaccagggaaagcccctaag

SEQ ID NO: 77 - DNA, 47 bp, Artificial
  Y36 DOM7h-8 Primer
cttaggggctttccctggtttctgctgmnnccaatttaaatagctgc

SEQ ID NO: 78 - DNA, 45 bp, Artificial
  L46 DOM7h-8 Primer
ccagggaaagcccctaagnnkctgatctatcggaattcccctttg

SEQ ID NO: 79 - DNA, 45 bp, Artificial
  L46 DOM7h-8 Primer
caaaggggaattccgatagatcagmnncttaggggctttccctgg

SEQ ID NO: 80 - DNA, 41 bp, Artificial
  Y87 DOM7h-8 Primer
cctgaagattttgctacgtacnnktgtcaacagacgtatag

SEQ ID NO: 81 - DNA, 41 bp, Artificial
  Y87 DOM7h-8 Primer
ctatacgtctgttgacamnngtacgtagcaaaatcttcagg

SEQ ID NO: 82 - DNA, 41 bp, Artificial
  Y87C DOM7h-8 Primer
cctgaagattttgctacgtactgctgtcaacagacgtatag

SEQ ID NO: 83 - DNA, 41 bp, Artificial
  Y87C DOM7h-8 Primer
ctatacgtctgttgacagcagtacgtagcaaaatcttcagg

SEQ ID NO: 84 - DNA, 41 bp, Artificial
  Y87E DOM7h-8 Primer
cctgaagattttgctacgtacgaatgtcaacagacgtatag

SEQ ID NO: 85 - DNA, 41 bp, Artificial
  Y87E DOM7h-8 Primer
ctatacgtctgttgacattcgtacgtagcaaaatcttcagg

SEQ ID NO: 86 - DNA, 41 bp, Artificial
  Y87N DOM7h-8 Primer
cctgaagattttgctacgtacaactgtcaacagacgtatag

SEQ ID NO: 87 - DNA, 41 bp, Artificial
  Y87N DOM7h-8 Primer
ctatacgtctgttgacagttgtacgtagcaaaatcttcagg

SEQ ID NO: 88 - DNA, 41 bp, Artificial
  Y87D DOM7h-8 Primer
cctgaagattttgctacgtacgattgtcaacagacgtatag

SEQ ID NO: 89 - DNA, 41 bp, Artificial
  Y87D DOM7h-8 Primer
ctatacgtctgttgacaatcgtacgtagcaaaatcttcagg

SEQ ID NO: 90 - DNA, 41 bp, Artificial
  Y87K DOM7h-8 Primer
cctgaagattttgctacgtacaaatgtcaacagacgtatag

SEQ ID NO: 91 - DNA, 41 bp, Artificial
  Y87K DOM7h-8 Primer
ctatacgtctgttgacatttgtacgtagcaaaatcttcagg

SEQ ID NO: 92 - DNA, 41 bp, Artificial
  Y87P DOM7h-8 Primer
cctgaagattttgctacgtacccatgtcaacagacgtatag

SEQ ID NO: 93 - DNA, 41 bp, Artificial
  Y87P DOM7h-8 Primer
ctatacgtctgttgacacgggtacgtagcaaaatcttcagg

SEQ ID NO: 94 - DNA, 45 bp, Artificial
  L46C DOM7h-8 Primer
ccagggaaagcccctaagtgcctgatctatcggaattcccctttg

SEQ ID NO: 95 - DNA, 45 bp, Artificial
  L46C DOM7h-8 Primer
caaaggggaattccgatagatcaggcacttaggggctttccctgg

SEQ ID NO: 96 - DNA, 47 bp, Artificial
  Y36D DOM7h-8 Primer
gcagctatttaaattgggatcagcagaaaccagggaaagcccctaag

SEQ ID NO: 97 - DNA, 47 bp, Artificial
  Y36D DOM7h-8 Primer
cttaggggctttccctggtttctgctgatcccaatttaaatagctgc

SEQ ID NO: 98 - DNA, 47 bp, Artificial
  Y36N DOM7h-8 Primer
gcagctatttaaattggaaccagcagaaaccagggaaagcccctaag

SEQ ID NO: 99 - DNA, 47 bp, Artificial
  Y36N DOM7h-8 Primer
cttaggggctttccctggtttctgctggttccaatttaaatagctgc

SEQ ID NO: 100 - DNA, 47 bp, Artificial
  Y36M DOM7h-8 Primer
gcagctatttaaattggatgcagcagaaaccagggaaagcccctaag

SEQ ID NO: 101 - DNA, 47 bp, Artificial
  Y36M DOM7h-8 Primer
cttaggggctttccctggtttctgctgcatccaatttaaatagctgc

SEQ ID NO: 102 - DNA, 39 bp, Artificial
  A43I DOM7h-11-15 Primer
cagcagaaaccagggaaaattcctaagctcctgatcctt

SEQ ID NO: 103 - DNA, 39 bp, Artificial
  A43I DOM7h-11-15 Primer
aaggatcaggagcttaggaattttccctggtttctgctg

SEQ ID NO: 104 - DNA, 39 bp, Artificial
  A43D DOM7h-11-15 Primer
cagcagaaaccagggaaagatcctaagctcctgatcctt

SEQ ID NO: 105 - DNA, 39 bp, Artificial
  A43D DOM7h-11-15 Primer
aaggatcaggagcttaggatctttccctggtttctgctg

SEQ ID NO: 106 - DNA, 324 bp, Artificial
  DOM7h-11-15 dAb
gacatccagatgacccagtctccatcctccctgtctgcatctgtaggagaccgtgtcaccatcacttgccgggcaagtcgtccgattgggacgatgttaagttggtaccagcagaaaccagggaaagcccctaagctcctgatccttgctttttcccgtttgcaaagtggggtcccatcacgtttcagtggcagtggatctgggacagatttcactctcaccatcagcagtctgcaacctgaagattttgctacgtactactgcgcgcaggctgggacgcatcctacgacgttcggccaagggaccaaggtggaaatcaaacgg

SEQ ID NO: 107 - protein, 108 aa, Artificial
  DOM7h-11-15 dAb
DIQMTQSPSSLSASVGDRVTITCRASRPIGTMLSWYQQKPGKAPKLLILAFSRLQSGVPSRFSGSGSGTDFTLTISSLQPEDFATYYCAQAGTHPTTFGQGTKVEIKR

SEQ ID NO: 108 - DNA, 39 bp, Artificial
  A43I DOM7h-14-10 Primer
cagcagaaaccagggaaaattcctaagctcctgatcatg

SEQ ID NO: 109 - DNA, 39 bp, Artificial
  A43I DOM7h-14-10 Primer
catgatcaggagcttaggaattttccctggtttctgctg

SEQ ID NO: 110 - DNA, 39 bp, Artificial
  A43D DOM7h-14-10 Primer
cagcagaaaccagggaaagatcctaagctcctgatcatg

SEQ ID NO: 111 - DNA, 39 bp, Artificial
  A43D DOM7h-14-10 Primer
catgatcaggagcttaggatctttccctggtttctgctg

SEQ ID NO: 112 - DNA, 324 bp, Artificial
  DOM7h-14-10 dAb
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtttgaggcatcctaagacgttcggccaagggaccaaggtggaaatcaaacgg

SEQ ID NO: 113 - protein, 108 aa, Artificial
  DOM7h-14-10 dAb
DIQMTQSPSSLSASVGDRVTITCRASQWIGSQLSWYQQKPGKAPKLLIMWRSSLQSGVPSRFSGSGSGTDFTLTISSLQPEDFATYYCAQGLRHPKTFGQGTKVEIKR